SEQUENCE LISTING (3 sequences)

SEQ ID NO: 1 - DNA, 17 bp, Artificial Sequence
  Synthetic
agttatctacacgacgg

SEQ ID NO: 2 - DNA, 17 bp, Artificial Sequence
  Synthetic
ggcgtactattcactct

SEQ ID NO: 3 - DNA, 25 bp, Artificial Sequence
  Synthetic
gcgtcagacccctatctataaactc

(19) United States
(12) Patent Application Publication
(10) Pub. No.: US 2011/0165610 A1
(43) Pub. Date: Jul. 7, 2011

(54) COMPOSITIONS, SYSTEMS, AND METHODS FOR PRESERVATION AND/OR STABILIZATION OF A CELL AND/OR MACROMOLECULE

(76) Inventor: Tony Baker, Sonora, CA (US)

(21) Appl. No.: 13/016,706

(22) Filed: Jan. 28, 2011

Related U.S. Application Data
(63) Continuation of application No. 12/048,961, filed on Mar. 14, 2008, now abandoned.
Application No. 12/048,961 is a continuation-in-part of application No. 11/686,169, filed on Mar. 14, 2007, now abandoned.
Application No. 11/686,169 is a continuation-in-part of application No. PCT/US07/63982, filed on Mar. 14, 2007.
(60) Provisional application No. 60/894,795, filed on Mar. 14, 2007.
(60) Provisional application No. 60/970,881, filed on Sep. 7, 2007.
(60) Provisional application No. 60/983,468, filed on Oct. 29, 2007.

(51) Int. Cl. C12Q 1/02; C12N 5/07; C12N 1/04; C12N 5/04; C12N 1/16; C12N 1/20; C12N 5/078; C12N 5/071; C12N 9/24; C09K 15/02; C09K 15/06; C09K 15/22; B65D 90/06

(52) U.S. Cl. 435/29; 435/374; 435/243; 435/420; 435/255.1; 435/252.1; 435/200; 435/260; 435/366; 252/400.1; 252/400.62; 252/403; 206/524.3

(57) ABSTRACT

The present disclosure relates to compositions, systems, and methods for preserving and/or stabilizing a cell (e.g., a whole cell). A cell and/or macromolecule stabilizing composition may include a chelator, a chelator enhancing component, and optionally a base (e.g., a purine base or a pyrimidine base). A cell stabilizing method may include contacting a cell with a cell and/or macromolecule stabilizing composition. A cell stabilizing system may include a container suitable for receiving a sample containing a cell and a cell and/or macromolecule stabilizing composition. A cell may be preserved and/or stabilized under ambient conditions (e.g., without refrigeration). A cell may include a protein, a nucleic acid, and/or another biomolecule marker of cell preservation and/or stabilization. A composition may be configured to preserve and/or stabilize one or more cells for analysis by flow cytometry and simultaneously preserve and/or stabilize one or more intracellular nucleic acids for molecular analysis.

### CROSS-REFERENCE TO RELATED APPLICATIONS

This application claims the benefit of U.S. Provisional Patent Application Ser. No. 60/894,795, filed Mar. 14, 2007, U.S. Provisional Patent Application Ser. No. 60/970,881, filed Sep. 7, 2007, and U.S. Provisional Patent Application Ser. No. 60/983,468, filed Oct. 29, 2007. This application is a continuation-in-part of U.S. application Ser. No. 11/686,169 filed Mar. 14, 2007. This application is a continuation-in-part of International PCT Application No. PCT/US07/63982 filed Mar. 14, 2007. The contents of each of the foregoing applications is hereby incorporated in their entirety by reference.

### FIELD OF THE INVENTION

The present disclosure relates in general to compositions, systems, and methods for the preservation of a macromolecule and/or a biomolecule. For example, compositions, systems, and methods of the disclosure may be used to preserve and/or stabilize a macromolecule and/or a biomolecule in a condition in which it may interact with another molecule in a conformation-specific and/or sequence specific manner.

### BACKGROUND

Macromolecules and biomolecules may be unstable under some conditions. A nucleic acid molecule, for example, may be degraded in the presence of a nuclease. Similarly, a protein molecule may be degraded in the presence of a protease. Degradation of macromolecules and biomolecules may increase with time. The efficacy of assays that include detection of a property of such molecules (presence, concentration, sequence, conformation) may be reduced or lost where such degradation occurs. For example, a diagnostic or forensic assay that depends on detection of minute quantities of a biomolecule may be unable to return a reliable result where the biomolecule has been degraded.

Sexually-transmitted disease (STD) clinics regularly screen and treat patients for such diseases as gonorrhea and Syphilis. Infectious agents such as gonococci may be detected by analyzing a DNA sample. A genetic transformation test (GTT), such as Gonostat™ (Sierra Diagnostics, Inc., Sonora, Calif.), may be used to detect gonococcal DNA in specimens taken from the urethra of men, and the cervix and anus of women, according to H W Jaffe et al. (J. Inf. Dis. 146:275-279 (1982)). W L Whittington et al. obtained similar results (Abstr. Ann. Meeting Am. Soc. Microbiol., p. 315 (1983)). However, it is not always possible to immediately test a patient for the presence of an infectious agent. For example, clinical laboratories are not readily found in many rural or underdeveloped areas. In such circumstances, it is necessary to transport patient test specimens to a laboratory for analysis, during which time the target of interest may be partially or wholly degraded.

Degradation of a macromolecule and/or biomolecule may be reduced by lowering the temperature of the macromolecule or biomolecule. However, this option may not be available in all situations or it may not be available for a sufficiently long period of time (e.g., from the time of sample collection to the time of analysis). For example, where a sample is collected (e.g., from a patient) in a remote location, it may be difficult or impossible to preserve the target molecule long enough for the sample to be transported to a facility where the sample is analyzed. In addition, cooling may not be uniform across all samples and/or may not be consistent from experiment to experiment.

Degradation of a macromolecule and/or biomolecule may be reduced by heating a composition to a temperature sufficient to inactivate one or more nucleases or proteases. However only a limited number of proteases and nucleases are inactivated by heating. In addition, heating may degrade rather than preserve a target molecule.

Diagnosis of a disease may depend upon the condition of a cell being maintained between collection and analysis. However, like macromolecules, a cell (e.g., a whole cell) may be labile outside of its normal milieu. For example, cells (e.g., blood cells) removed from a human body may begin to deteriorate (e.g., lyse, oxidize, and/or coagulate) within seconds to minutes after removal.

### SUMMARY

Therefore, a need has arisen for compositions, systems, and methods for preserving and/or stabilizing a cell (e.g., whole cell).

The present disclosure relates to compositions, systems, and methods for preserving and/or stabilizing a macromolecule and/or biomolecule (collectively, “macromolecule”). According to some embodiments, a macromolecule and/or a biomolecule may include a protein and/or a nucleic acid (e.g., DNA and RNA). As will be appreciated by those of ordinary skill in the art, a nucleic acid may include sequences from a plurality of sources. For example, a single nucleic acid may include an artificial sequence (e.g., a primer binding site), a human sequence (e.g., adenomatous polyposis coli (APC), amyloid precursor protein (APP), breast cancer 1 (BRCA1), transmembrane protease serine 2 (TMPRSS2), v-ets erythroblastosis virus E26 oncogene homolog (ERG)), a plant sequence, a microbial sequence (e.g., an antibiotic resistance gene), a viral sequence (e.g., HIV protease), and/or combinations thereof. A single nucleic acid sequence may also include an unusual or artificial fusion of two sequences from a common source (e.g., a TMPRSS2:ERG fusion). A macromolecule may be regarded as preserved as long as the macromolecule, if present, is maintained in a detectable form at least from the time of sample collection to the time of sample analysis. In some embodiments, the disclosure relates to preservation and/or stabilization of macromolecules in a bodily fluid or excretion (e.g., urine, blood, blood serum, amniotic fluid, spinal fluid, conjunctival fluid, salivary fluid, vaginal fluid, stool, seminal fluid, and sweat). In some embodiments, an unexpected improvement in nucleic acid hybridization may be observed in such nucleic acid testing methods (e.g., compared with the same methods practiced in the absence of a preservation composition, system, or method of the disclosure).

The present disclosure relates to compositions, systems, and methods for preserving and/or stabilizing a cell (e.g., whole cell) and/or a macromolecule and/or a biomolecule (collectively, “macromolecule”). For example, the present disclosure, according to some embodiments, relates to a composition for preserving and/or stabilizing a cell (e.g., whole cell) (a “cell stabilizing composition”).

In some embodiments, a cell and/or macromolecule stabilizing composition may include (a) a chelator (e.g., a chelator selected from the group consisting of ethylenediaminetetraacetic acid (EDTA), [ethylenebis(oxyethylenenitrilo)]tetraacetic acid (EGTA), 1,2-bis(2-aminophenoxy)ethane-N,N,N′,N′-tetraacetic acid (BAPTA), and salts thereof), (b) at least one chelator enhancing component (e.g., a chelator enhancing component selected from the group consisting of guanidine, lithium chloride, sodium salicylate, sodium perchlorate, and sodium thiocyanatc), and optionally (c) a base selected from the group consisting of a purine base and a pyrimidine base. The concentration of a chelator, a chelator enhancing component, and/or a base may be each selected from any attainable concentration. For example, the concentration of a chelator may be from about 0.1 mM to about 0.1 M, the concentration of a chelator enhancing component may be from about 1 mM to about 5 M; and/or the concentration of a base may be from about 0.1 mM to about 5 M. In some embodiments, a cell and/or macromolecule stabilizing composition may be formulated as an aqueous solution. In some embodiments, a chelator enhancing component may be selected from the group consisting of sodium perchlorate, sodium thiocyanate, and lithium chloride. A cell and/or macromolecule stabilizing composition may include or exclude, according to some embodiments, at least one enzyme inactivating component selected from the group consisting of manganese chloride, sarkosyl, sodium dodecyl sulfate, and combinations thereof. In some embodiments, a cell and/or macromolecule stabilizing composition may include a solid, a liquid, and/or a hydrogel. A cell and/or macromolecule stabilizing composition may include a solvent (e.g., water and/or an organic solvent) in some embodiments. A cell and/or macromolecule stabilizing composition may include, according to some embodiments, a cell (e.g., an intact, a whole cell, and the like), a protein (e.g., a cellular and/or cell-free protein), and/or a nucleic acid (e.g., a cellular and/or cell-free RNA, DNA, and the like).

In some embodiments, a cell and/or macromolecule stabilizing composition may include a plasticizer (e.g., a citrated alcohol) and/or an anticoagulant (e.g., heparin). A cell and/or macromolecule stabilizing composition may reduce and/or block clumping and/or coagulation in samples (e.g., blood samples) stored at room temperature (e.g., about 20° C.) in some embodiments.

A cell and/or macromolecule stabilizing composition may include a buffer according to some embodiments. For example, a buffer may include a compound selected from the group consisting of potassium acetate, sodium acetate, potassium phosphate, sodium phosphate, tris(hydroxyamino)methane, N-(2-hydroxyethyl)piperazine-N′-(2-ethanesulfonic acid), 3-(N-morpholino)propane sulfonic acid, 2-[(2-amino-2-oxoethyl)amino]ethanesulfonic acid, N-(2-acetamido)2-iminodiacetic acid, 3-[(1,1-dimethyl-2-hydroxyethyl)amino]-2-propanesulfonic acid, N,N-bis(2-hydroxyethyl)-2-aminoethanesulfonic acid, N,N-bis(2-hydroxyethylglycine, bis-(2-hydroxyethyl)imino-tris(hydroxymethyl)methane, 3-(cyclohexylamino)-1-propanesulfonic acid, 3-(cyclohexylamino)-2-hydroxy-1-propanesulfonic acid, 2-(N-cyclohexylamino)ethanesulfonic acid, 3-[N,N-bis(2-hydroxyethyl)amino]-2-hydroxy-propanesulfonic acid, N-(2-hydroxyethylpiperazine)-N′-(3-propanesulfonic acid), N-(2-hydroxyethyl)piperazine-N′-(2-hydroxypropanesulfonic acid), 2-(N-morpholine)ethanesulfonic acid, triethanolamine buffer, imidazole, glycine, ethanolamine, 3-(N-morpholine)-2-hydroxypropanesulfonic acid, piperazine-N,N′-bis(2-ethanesulfonic acid), piperazine-N,N′-bis(2-hydroxypropanesulfonic acid), N-tris[(hydroxymethyl)methyl]-3-aminopropanesulfonic acid, 2-hydroxy-3-[tris(hydroxymethyl)methylamino]-1-propanesulfonic acid, N-[Tris(hydroxymethyl)methyl]-2-aminoethanesulfonic acid, N-[Tris(hydroxymethyl)methyl]glycine, 2-amino-2-methyl-1,3-propanediol, 2-amino-2-methyl-1-propanol, and combinations thereof.

According to some embodiments, the present disclosure also relates to a method of preserving and/or stabilizing a cell (a “cell stabilizing method”). A cell stabilizing method may include, for example, contacting a macromolecule with a cell and/or macromolecule stabilizing composition comprising (a) a chelator (e.g., a chelator selected from the group consisting of ethylenediaminetetraacetic acid (EDTA), [ethylenebis(oxyethylenenitrilo)]tetraacetic acid (EGTA), 1,2-bis(2-aminophenoxy)ethane-N,N,N′, N′-tetraacetic acid (BAPTA), and salts thereof), (b) at least one chelator enhancing component (e.g., a chelator enhancing component selected from the group consisting of guanidine, lithium chloride, sodium salicylate, sodium perchlorate, and sodium thiocyanate,) and (c) a base (e.g., a base selected from the group consisting of a purine base and a pyrimidine base). A cell, in some embodiments, may include a cell selected from the group consisting of a mammalian cell, a plant cell, a yeast cell, a bacterial cell, a virally-infected cell, a diseased cell, and combinations thereof. In some embodiments, a mammalian cell may include a cell selected from the group consisting of an erythrocyte, a leukocyte, a lymphocyte, a histiocyte, an epithelial cell, and combinations thereof. A mammalian cell may include a human cell according to some embodiments. A macromolecule to be preserved and/or stabilized with a macromolecule stabilizing composition and/or method may include, according to some embodiments, a nucleic acid selected from the group consisting of DNA, RNA, mRNA, and cDNA. A nucleic acid may include, for example, prokaryotic and/or eukaryotic DNA. In some embodiments, a cell and/or macromolecule to be preserved and/or stabilized with a cell and/or macromolecule stabilizing composition and/or method may be present in a bodily fluid obtained from a human subject. A bodily fluid may include, for example, a material selected from the group consisting of blood, blood serum, amniotic fluid, spinal fluid, conjunctival fluid, salivary fluid, vaginal fluid, stool, seminal fluid, and sweat.

The present disclosure further relates to a system for preserving and/or stabilizing a cell (e.g., whole cell) (a “cell stabilizing system”) and/or a macromolecule (a “macromolecule stabilizing system”) in some embodiments. A cell and/or macromolecule stabilizing system may include a sample container configured and arranged to receive and contain a sample comprising a cell and a cell and/or macromolecule stabilizing composition (e.g., including a chelator, at least one chelator enhancing component, and a base). A system may also include user instructions in some embodiments. The sample container, in some embodiments, may contain the cell and/or macromolecule stabilizing composition. For example, the sample container may include at least one inner surface and at least one outer surface with a cell and/or macromolecule stabilizing composition coated onto the latter. A sample container may include at least one vesicle, liposome, and/or micelle in some embodiments. A cell and/or macromolecule stabilizing composition may be present within the lumen of a vesicle, liposome, and/or micelle.

### BRIEF DESCRIPTION OF THE DRAWINGS

Some embodiments of the disclosure may be understood by referring, in part, to the following description and the accompanying drawings, wherein:

FIG. 1 is a bar graph of DNA concentration in preserved urine according to an embodiment of the disclosure;

FIG. 2 is a graph of eight day serial data on preserved urine according to an embodiment of the disclosure;

FIG. 3 is a graph comparing PCR results in unpreserved and preserved normal urine according to an embodiment of the disclosure;

FIG. 4 is a graph of eight day serial data on preserved serum according to an embodiment of the disclosure;

FIG. 5 is a graph of DNA concentration in preserved serum according to an embodiment of the disclosure;

FIG. 6 is a diagram of the system for preserving DNA according to one embodiment of the disclosure;

FIG. 7 graphically illustrates a comparison of signal response in PCR assays wherein the DNA has been treated with a preservative of the disclosure, and one which has not;

FIG. 8 illustrates the efficacy of reagents of the present disclosure to enhance signal response of a branched DNA assay of blood plasma samples subjected to various storage conditions;

FIG. 9 illustrates the efficacy of reagents of the present disclosure to enhance signal response of a branched DNA assay of blood serum and plasma samples;

FIG. 10 is a graph showing the interference of methemoglobin on PCR absorbance in a PCR amplification assay on hepatitis B sequences MD03/06 in unprotected serum;

FIG. 11 is a graph showing the improvement in attenuating the interference of methemoglobin on PCR absorbance in a PCR amplification assay on hepatitis B sequences MD03/06 in serum which has been treated with a preservative of the disclosure;

FIG. 12A is a chart showing a representation of results obtained from an example PCR amplification using MD03 and MD06 primers and a hepatitis B template in serum contacted with buffer (no protection), guanidine only, EGTA only, or EGTA+guanidine;

FIG. 12B is a chart showing a representation of results obtained from an example PCR amplification using MD03 and MD06 primers and a hepatitis B template in serum contacted with buffer (no protection), EDTA only, sodium perchlorate only, or EDTA+sodium perchlorate;

FIG. 12C is a chart showing a representation of results obtained from an example PCR amplification using MD03 and MD06 primers and a hepatitis B template in serum contacted with buffer (no protection), EGTA only, sodium perchlorate only, or EGTA+sodium perchlorate;

FIG. 12D is a chart showing a representation of results obtained from an example PCR amplification using MD03 and MD06 primers and a hepatitis B template in serum contacted with buffer (no protection), EDTA only, or EDTA+sodium thiocyanate;

FIG. 12E is a chart showing a representation of results obtained from an example PCR amplification using MD03 and MD06 primers and a hepatitis B template in serum contacted with buffer (no protection), EGTA only, or EGTA+sodium thiocyanate;

FIG. 12F is a chart showing a representation of results obtained from an example PCR amplification using MD03 and MD06 primers and a hepatitis B template in serum contacted with buffer (no protection) or BAPTA only;

FIGS. 13A-13G are graphs showing the absence of preservative effect on gonococcal DNA in urine stored at room temperature and subsequently subjected to PCR detection offered by the individual addition of certain components which are included in the reagents of the disclosure;

FIG. 14A is a chart showing the results of an example PCR amplification using a gonococcal DNA template in fresh urine contacted with cytosine only or sodium thiocyanate+EDTA+cytosine;

FIG. 14B is a chart showing the results of an example PCR amplification using a gonococcal DNA template in fresh urine contacted with guanine only or sodium thiocyanate+EDTA+guanine;

FIG. 14C is a chart showing the results of an example PCR amplification using a gonococcal DNA template in fresh urine contacted with thymine only or sodium thiocyanate+EDTA+thymine;

FIG. 14D is a chart showing the results of an example PCR amplification using a gonococcal DNA template in fresh urine contacted with uracil only or sodium thiocyanate+EDTA+uracil;

FIG. 15A is a chart showing the results of an example PCR amplification using a gonococcal DNA template in fresh urine contacted with 1 M adenine, 1 M sodium thiocyanatc, 1 M EDTA, or 1 M sodium thiocyanate+0.01 M EDTA+1 M adenine;

FIG. 15B is a chart showing the results of an example PCR amplification using a gonococcal DNA template in fresh urine contacted with 1 M adenine, 1 M EDTA, 2 M sodium thiocyanate+1 M EDTA, or 2 M sodium thiocyanate+1 M EDTA+1 M adenine;

FIG. 15C is a chart showing the results of an example PCR amplification using a gonococcal DNA template in fresh urine contacted with 1 M adenine, 1 M guanidine, 1 M guanidine+0.01 M EDTA, 2 M sodium thiocyanate+1 M EGTA, or 1 M Guanidine.HCl+1 M EGTA+2 M adenine;

FIG. 15D is a chart showing the results of an example PCR amplification using a gonococcal DNA template in fresh urine contacted with 1 M adenine, 1 M guanidine, 1 M guanidine+0.01 M EDTA, 1 M lithium chloride+1 M BAPTA, or 2 M guanidine thiocyanate+1 M BAPTA+2 M adenine;

FIG. 15E is a chart showing the results of an example PCR amplification using a gonococcal DNA template in fresh urine contacted with 1 M sodium perchlorate, 1 M sodium thiocyanate+2 M EDTA, or 1 M sodium perchlorate+1 M EDTA;

FIG. 16A is a chart showing the results of an example PCR amplification using a gonococcal DNA template in fresh urine contacted with 1 M guanidine.HCl or 1 M guanidine.HCl+0.01 M BAPTA+4 M adenine;

FIG. 16B is a chart showing the results of an example PCR amplification using a gonococcal DNA template in fresh urine contacted with 0.01 M EDTA, 2 M sodium thiocyanate, 1 M sodium thiocyanate+0.1 M EDTA+1 M adenine, or 2 M sodium thiocyanate+0.1 M EGTA+2 M adenine;

FIG. 17A is a plot of CD3 percentage over time for cells contacted with a test composition (e.g., an example embodiment of the disclosure or a control) and subjected to flow cytometry analysis;

FIG. 17B is a plot of CD4 percentage over time for cells contacted with a test composition (e.g., an example embodiment of the disclosure or a control) and subjected to flow cytometry analysis;

FIG. 17C is a plot of absolute CD3 count over time for cells contacted with a test composition (e.g., an example embodiment of the disclosure or a control) and subjected to flow cytometry analysis;

FIG. 18 is a plot of total RNA yield (measured area under the curve) over time for samples contacted with a test composition (e.g., an example embodiment of the disclosure or a control);

FIG. 19A is an electropherogram of an RNA-containing sample contacted with a PAXgene™ composition;

FIG. 19B is an electropherogram of an RNA-containing sample contacted with an EDTA composition;

FIG. 19C is an electropherogram of an RNA-containing sample contacted with a composition according to a specific example embodiment of the disclosure; and

FIG. 19D is an electropherogram of an RNA-containing sample contacted with a composition according to a specific example embodiment of the disclosure.

### DESCRIPTION

The present disclosure relates to compositions, systems, and methods for delaying degradation of a cell (e.g., whole cell) and/or a macromolecule and/or a biomolecule (“macromolecule”).

According to some embodiments, a composition may preserve and/or stabilize a cell (a “cell and/or macromolecule stabilizing composition”). A cell may include a whole cell. A whole cell may include, for example, cell surface materials (e.g., cell surface proteins, extracellular matrix, cell wall, and/or outer membrane). A cell that may be preserved and/or stabilized may include a cell selected from a mammalian cell (e.g., a human cell), a plant cell, a yeast cell, a bacterial cell, a virally-infected cell, a diseased cell, and combinations thereof. A mammalian cell may include a cell selected from an erythrocyte, a leukocyte, a lymphocyte, a histiocyte, an epithelial cell, a stem cell, and combinations thereof.

A cell may be preserved and/or stabilized, in some embodiments, where it is kept alive. In some embodiments, a cell may be preserved and/or stabilized where it is maintained in the same or substantially the same condition (e.g., morphologically, physiologically, genetically, and/or biochemically) as an in vivo cell. A cell may be preserved and/or stabilized, in some embodiments, where it is kept in the same or substantially the same condition (e.g., healthy or diseased) as it was when it was in a body or a bodily fluid. For example, preservation and/or stabilization may be assessed in terms of the energy consumption of a cell, the amount of a metabolite present (e.g., pyruvate), the amount of free ATP present, the rate of transcription and/or translation, and/or the presence (or absence) of one or more proteins and/or nucleic acids.

According to some embodiments, a macromolecule and/or a biomolecule may include a protein and/or a nucleic acid (e.g., DNA and RNA). As will be appreciated by those of ordinary skill in the art, a nucleic acid may include sequences from a plurality of sources. For example, a single nucleic acid may include an artificial sequence (e.g., a primer binding site), a human sequence (e.g., adenomatous polyposis coli (APC), amyloid precursor protein (APP), breast cancer 1 (BRCA1), transmembrane protease serine 2 (TMPRSS2), v-ets erythroblastosis virus E26 oncogene homolog (ERG)), a plant sequence, a microbial sequence (e.g., an antibiotic resistance gene), a viral sequence (e.g., HIV protease), and/or combinations thereof. A single nucleic acid sequence may also include an unusual or artificial fusion of two sequences from a common source (e.g., a TMPRSS2:ERG fusion). A macromolecule may be regarded as preserved as long as the macromolecule, if present, is maintained in a detectable form at least from the time of sample collection to the time of sample analysis. In some embodiments, the disclosure relates to preservation and/or stabilization of macromolecules in a bodily fluid or excretion (e.g., urine, blood, blood scrum, amniotic fluid, spinal fluid, conjunctival fluid, salivary fluid, vaginal fluid, stool, seminal fluid, and sweat). In some embodiments, an unexpected improvement in nucleic acid hybridization may be observed in such nucleic acid testing methods (e.g., compared with the same methods practiced in the absence of a preservation composition, system, or method of the disclosure).

Degradation may be regarded as any change in molecular structure that renders undetectable a molecule of interest or a collection of molecules of interest. For example, degradation of a protein may include any modification of the primary, secondary, tertiary or quaternary structure (e.g., reduction of disulfide bonds, hydrolysis of peptide bonds, or any other cleavage of a covalent, ionic, hydrophobic, hydrogen, or Van der Waals bond). Degradation of a nucleic acid may include any modification of the hybridization state (e.g., single, double, or triple stranded), helical structure (e.g., A, B, or Z), supercoiling, or sequence (e.g., pyrimidine dimerization, deamination, oxidation, depurination, or any other cleavage of a covalent, ionic, hydrophobic, or hydrogen bond). This delay in degradation may be regarded as preserving the macromolecule in a desired form for a long or indefinite period of time. This delay may also be regarded as preserving or stabilizing the macromolecule in a desired form for a defined period (e.g., from the time of sample collection to the time of assay).

Compositions, systems, and methods according to some embodiments of the disclosure may reduce or eliminate degradation of a macromolecule in a biological fluid and/or excretion. For example, a composition, system, and/or method of the disclosure may, in some embodiments, eliminate enzymatic destruction of a nucleic acid of interest in a bodily fluid (e.g., urine). Nucleic acids that may be preserved and/or stabilized include, for example natural and/or synthetic forms of DNA, RNA, RNA/DNA hybrids, and variants thereof. Nucleic acids that may be preserved and/or stabilized may include an intercellular nucleic acid and/or an intracellular nucleic acid. DNA that may be preserved and/or stabilized may include, for example, human DNA, mammalian DNA, bacterial DNA, fungal DNA, and viral DNA. Bacterial DNA that may be preserved and/or stabilized may include, for example, gonococcal DNA, Haemophilus influenzae DNA, and Bacillus subtilis DNA.

A cell and/or a macromolecule (and/or biomolecule) to be preserved and/or stabilized may be comprised in a bodily fluid and/or excretion, a tissue (e.g., biopsy tissue), and/or an object (e.g., bone). For example, a macromolecule may be comprised in a food particle, a soil sample, a forensic sample (e.g., an article of clothing, a hair, a finger print), a fabric, a bacterial matrix, a slime, an environmental specimen, and/or a biowarfare specimen. A macromolecule (and/or biomolecule) to be preserved and/or stabilized may be comprised in a whole cell and/or purified (e.g., fully or partially purified) from a whole cell.

Compositions, systems, and methods may preserve and/or stabilize a cell and/or a macromolecule (e.g., at room temperature) for at least about 1 day, at least about 2 days, at least about 3 days, at least about 4 days, at least about 5 days, at least about 6 days, at least about a week, at least about 2 weeks, at least about 3 weeks, and/or at least about 4 weeks. Compositions, systems, and methods may preserve and/or stabilize a cell and/or a macromolecule (e.g., at room temperature) for up to about 1 day, up to about 2 days, up to about 3 days, up to about 4 days, up to about 5 days, up to about 6 days, up to about a week, up to about 2 weeks, up to about 3 weeks, and/or up to about 4 weeks. Compositions, systems, and methods, in some embodiments, may preserve and/or stabilize a cell and/or a macromolecule for any of the foregoing periods without refrigeration. For example, preservation and/or stabilization may be achieved where the ambient temperature and/or temperature of the composition does not exceed about 70° C., about 60° C., about 55° C., about 50° C., about 45° C., and/or about 40° C. Preservation and/or stabilization may be achieved where the ambient temperature and/or temperature of the composition is from about 0° C. to about 10° C., from about 10° C. to about 20° C., from about 15° C. to about 25° C., from about 20° C. to about 30° C., from about 15° C. to about 35° C., and/or from about 30° C. to about 40° C. The choice of temperature range, in some embodiments, may be chosen based on the expected and/or desired storage conditions for a specific sample. For example, compositions, systems, and methods may be adapted to preserving and/or stabilizing materials collected in an under developed country where refrigeration is impractical and/or unavailable and day time temperatures approach 50° C. Likewise, compositions, systems, and methods may be adapted to preserving and/or stabilizing materials collected in a location where shipping conditions, storage conditions, and/or ambient conditions include temperatures below 20° C.

Without being limited to any particular mechanism of action, compositions, systems, and methods of the disclosure may inactivate one or more metal-dependent enzymes and/or one or more metal-independent enzymes present in a test sample (e.g., bodily fluid) containing the macromolecule and/or biomolecule of interest. For example, a divalent metal chelator may bind available metals (e.g., Mg²⁺ and Ca²⁺) to such an extent that metals that remain available to the metal-dependent enzymes (e.g., deoxyribonucleases) are insufficient to support catalysis (i.e., nucleic acid degradation). Again, without being limited to any particular mechanism of action, a chelator enhancing component may inactivate one or more metal independent enzymes found in a bodily fluid. For example, a metal independent enzyme may include a DNA ligase (e.g., D4 DNA ligase), a DNA polymerase (e.g., T7 DNA polymerase), an exonuclease (e.g., exonuclease 2, λ-exonuclease), a kinase (e.g., T4 polynucleotide kinase), a phosphotase (e.g., BAP and CIP phosphotase), a nuclease (e.g., BL31 nuclease and XO nuclease), and an RNA-modifying enzyme (e.g., E. coli RNA polymerase, SP6, T7, T3 RNA polymerase, and T4 RNA ligase). Without being limited to any particular mechanism of action a purine base and/or a pyrimidine base may bind to a nucleic acid and act as an isomeric target for one or more enzymes that degrade DNA and/or RNA.

According to some specific example embodiments of the disclosure, the yield from PCR amplification of a target nucleic acid (e.g., gonococcal DNA) contacted with a cell and/or a macromolecule stabilizing composition having purine base may be at least about 2-fold higher, about 3-fold higher, about 4-fold higher, about 5-fold higher, about 6-fold higher, about 7-fold higher, about 8-fold higher, about 9-fold higher, and/or 10-fold higher than the yield from PCR amplification of the same target nucleic acid not contacted with a cell and/or a macromolecule stabilizing composition having a purine base. According to some specific example embodiments of the disclosure, the yield from PCR amplification of a target nucleic acid (e.g., gonococcal DNA) contacted with a cell and/or a macromolecule stabilizing composition having a chelator, a chelator enhancing component, and a purine base may be about 2-fold higher, about 3-fold higher, about 4-fold higher, about 5-fold higher, about 6-fold higher, about 7-fold higher, about 8-fold higher, about 9-fold higher, and/or 10-fold higher than the yield from PCR amplification of the same target nucleic acid contacted with a cell and/or a macromolecule stabilizing composition having a chelator and a chelator enhancing component, but lacking a purine base. For example, the yield from PCR amplification of a target nucleic acid (e.g., gonococcal DNA) contacted with a cell and/or a macromolecule stabilizing composition having EDTA (e.g., 0.1 M), sodium thiocyanate (e.g., 1 M), and adenine may be about 10-fold higher than the yield from PCR amplification of the same target nucleic acid contacted with a cell and/or a macromolecule stabilizing composition having EDTA (e.g., 0.1 M) and sodium thiocyanate (e.g., 1 M), but lacking adenine.

### Compositions

A composition for preserving and/or stabilizing a macromolecule and/or biomolecule (a “macromolecule stabilizing composition”), according to some embodiments of the disclosure may include a chelator, a chelator enhancing component, a purine base, and/or a pyrimidine base. For example, a macromolecule stabilizing composition may include a chelator, a chelator enhancing component, and a purine base. A composition for preserving and/or stabilizing a cell (e.g., a whole cell) (a “cell and/or macromolecule stabilizing composition”), according to some embodiments of the disclosure may include a chelator, a chelator enhancing component, a purine base, and/or a pyrimidine base. For example, a cell and/or macromolecule stabilizing composition may include a chelator, a chelator enhancing component, and a purine base.

A chelator may include, for example, ethylenediaminetetraacetic acid (EDTA), [ethylenebis(oxyethylenenitrilo)]tetraacetic acid (EGTA) and 1,2-bis(2-aminophenoxy)ethane-N,N,N′,N′-tetraacetic acid (BAPTA), and/or salts thereof. A chelator, if included, may be present at any desirable concentration. For example, a chelator may be included at a concentration of at least about 0.1 mM, at least about 0.005 M, at least about 0.01 M, at least about 0.05 M, and/or at least about 0.1 M. A chelator may be included at a concentration of up to about 0.1 mM, up to about 5 mM, up to about 0.01 M, up to about 0.05 M, and/or up to about 0.1 M. A chelator may be included at a concentration of from about 0.1 mM to about 0.1M. Where two or more chelators are included in a single composition, either the concentration of each chelator or the total concentration of the combined chelators may fall within any of the provided ranges. In some embodiments, a chelator may include EDTA, EGTA, BAPTA, imidazole, iminodiacetate (IDA), bis(5-amidino-2-benzimidazolyl)methane (BABIM), and/or salts thereof.

A chelator enhancing component may include, for example, lithium chloride, guanidine, sodium salicylate, sodium perchlorate, sodium thiocyanate, and combinations thereof. As those of ordinary skill in the art will appreciate, guanidine includes guanine, a purine base, and a ribose. A chelator enhancing component, if included, may be present at any desirable concentration. For example, a chelator enhancing component may be included at a concentration of at least about 1 mM, at least about 10 mM, at least about 0.05 M, at least about 0.1 M, at least about 0.5 M, at least about 1 M, at least about 1.5 M, at least about 1.75 M, at least about 2 M, at least about 3 M, at least about 4 M, and/or at least about 5 M. A chelator enhancing component may be included at a concentration of up to about 1 mM, up to about 0.05 M, up to about 0.1 M, up to about 0.5 M, up to about 1 M, up to about 1.5 M, up to about 1.75 M, and/or up to about 2 M. A chelator enhancing component may be present at a concentration within a range having endpoints defined by any of the foregoing concentrations. For example, a chelator enhancing component may be included at a concentration of from about 1 mM to about 0.5 M, from about 0.1 M to about 1.75 M, from about 0.1 M to about 2.0 M, from about 0.1 M to about 3.0 M, from about 0.5 M to about 3.0 M, and/or from about 0.1 M to about 5.0 M.

A purine base may include adenine, guanine, and combinations thereof. A purine base may also include analogs and/or variants (e.g., methyladenine, methylguanine, ethyladenine, ethylguanine). A purine base may also include structurally similar analogs and/or variants such as inosine, caffeine, uric acid, theobromine, theophylline, 2-aminopurine, 6-aminopurine, hypoxanthine (6-oxy purine), and xanthine (2,6-dioxy purine). A purine base may include a salt (e.g., adenine hemisulfate salt, adenine hydrochloride). A purine base, if included, may be present at any desirable concentration. For example, a purine base may be included at a concentration of at least about 0.1 mM, at least about 1 mM, at least about 10 mM, at least about 0.1 M, at least about 0.25 M, at least about 0.5 M, at least about 0.75 M, at least about 1 M, at least about 1.5 M, at least about 1.75 M, at least about 2 M, at least about 2.5 M, at least about 3 M, at least about 4 M, at least about 5 M, at least about 6 M, and/or at least about 7 M. A purine base may be included at a concentration of up to about 0.1 mM, up to about 1 mM, up to about 10 mM, up to about 0.1 M, up to about 0.25 M, up to about 0.5 M, up to about 0.75 M, up to about 1 M, up to about 1.5 M, up to about 2 M, up to about 2.5 M, up to about 3 M, up to about 4 M, up to about 5 M, up to about 6 M, and/or up to about 7 M. A purine base, if included, may be present at a concentration within a range having endpoints defined by any of the foregoing concentrations. For example, a purine base may be included at a concentration of from about 0.1 mM to about 100 mM, from about 1 mM to about 10 mM, from about 0.1 M to about 1.0 M, from about 0.1 M to about 2.0 M, from about 0.1 M to about 5.0 M, from about 0.1 M to about 1.75 M, from about 0.5 M to about 2.0 M, from about 0.75 M to about 3 M, and/or from about 0.1 M to about 7 M.

A pyrimidine base may include, for example, cytosine, thymine, uracil, and combinations thereof. A pyrimidine base may also include analogs and/or variants (e.g., methylcytosine, methylthymine, methyluracil, ethylcytosine, ethylthymine, ethyluracil). A pyrimidine base may also include structurally similar analogs and/or variants such as orotic acid, thiamine, 5-fluorouracil, 6-azauracil, pyrazine, and/or pyridazine. A pyrimidine base may include a salt (e.g., pyrimidine salt, 2-piperazinopyrimidine salt). A pyrimidine base, if included, may be present at any desirable concentration. For example, a pyrimidine base may be included at a concentration of at least about 0.1 mM, at least about 1 mM, at least about 10 mM, at least about 0.1 M, at least about 0.25 M, at least about 0.5 M, at least about 0.75 M, at least about 1 M, at least about 1.5 M, at least about 1.75 M, at least about 2 M, at least about 2.5 M, at least about 3 M, at least about 4 M, at least about 5 M, at least about 6 M, and/or at least about 7 M. A pyrimidine base may be included at a concentration of up to about 0.1 mM, up to about 1 mM, up to about 10 mM, up to about 0.1 M, up to about 0.25 M, up to about 0.5 M, up to about 0.75 M, up to about 1 M, up to about 1.5 M, up to about 2 M, up to about 2.5 M, up to about 3 M, up to about 4 M, up to about 5 M, up to about 6 M, and/or up to about 7 M. A pyrimidine base, if included, may be present at a concentration within a range having endpoints defined by any of the foregoing concentrations. For example, a pyrimidine base may be included at a concentration of from about 0.1 mM to about 100 mM, from about 1 mM to about 10 mM, from about 0.1 M to about 1.0 M, from about 0.1 M to about 2.0 M, from about 0.1 M to about 5.0 M, from about 0.1 M to about 1.75 M, from about 0.5 M to about 2.0 M, from about 0.75 M to about 3 M, and/or from about 0.1 M to about 7 M.

In some embodiments, a cell and/or a macromolecule stabilizing composition may include an amount of a divalent metal chelator selected from EDTA, EGTA BAPTA, and salts thereof; and an amount of at least one chelator enhancing component selected from lithium chloride, guanidine, sodium salicylate, sodium perchlorate, and sodium thiocyanate. The amount of a divalent metal chelator may be generally in the range of from about 0.1 mM to about 0.1 M. The amount of a chelator enhancing component may be generally in the range of from about 1 mM to about 500 mM. The amount of chelator in a composition may be, for example, at least about 0.01 M. The amount of chelator enhancing component in a composition may be, for example, at least about 1 M.

According to some embodiments, a macromolecule stabilizing composition may include an amount of at least one enzyme inactivating component such as manganese chloride, sarkosyl, or sodium dodecyl sulfate, generally in the range of about 0-5% molar concentration. In some embodiments, a cell and/or macromolecule stabilizing composition may include or exclude an enzyme inactivating component.

In some embodiments, a cell and/or a macromolecule stabilizing composition may include a purine base, a pyrimidine base, or both a purine base and a pyrimidine base. For example, a composition may include a chelator, a chelator enhancing component, and a purine base (e.g., adenine). In some embodiments, a cell and/or a macromolecule stabilizing composition may include only (a) a chelator, (b) a chelator enhancing component, and (c) a purine base and/or a pyrimidine base. A cell and/or a macromolecule stabilizing composition, in other embodiments, may include one or more solvents (e.g., aqueous and/or organic), buffers, salts, surfactants, oxidizing agents, reducing agents, and/or other reagents.

In some embodiments, a cell and/or a macromolecule stabilizing composition may have a pH of from about 4.5 to about 8.5. A cell and/or a macromolecule stabilizing composition may be formulated such that upon being combined with the sample to be preserved and/or stabilized (e.g., a bodily fluid), the mixture has a pH of from about 4.5 to about 8.5. In some embodiments, a suitable buffer may be selected from Good buffers (e.g., HEPES), potassium acetate, sodium phosphate, potassium bicarbonate, tris(hydroxyamino)methane (Tris), and combinations thereof. For example, a buffer may include potassium acetate, sodium acetate, potassium phosphate, sodium phosphate, Tris, N-(2-hydroxyethyl)piperazine-N′-(2-ethanesulfonic acid) (HEPES) buffer, 3-(N-morpholino)propane sulfonic acid (MOPS) buffer, 2-[(2-amino-2-oxoethyl)amino]ethanesulfonic acid (ACES) buffer, N-(2-acetamido)-2-iminodiacetic acid buffer (ADA), 3-[(1,1-dimethyl-2-hydroxyethyl)amino]-2-propanesulfonic acid (AMPSO) buffer, N,N-bis(2-hydroxyethyl)-2-aminoethanesulfonic acid (BES) buffer, Bicine (N,N-bis(2-hydroxyethylglycine) buffer, bis-(2-hydroxyethyl)imino-tris(hydroxymethyl)methane (Bis-Tris) buffer, 3-(cyclohexylamino)-1-propanesulfonic acid (CAPS) buffer, 3-(cyclohexylamino)-2-hydroxy-1-propanesulfonic acid (CAPSO) buffer, 2-(N-cyclohexylamino)ethanesulfonic acid (CHES) buffer, 3-[N,N-bis(2-hydroxyethyl)amino]-2-hydroxy-propanesulfonic acid (DIPSO) buffer, N-(2-hydroxyethylpiperazine)-N′-(3-propanesulfonic acid) (HEPPS) buffer, N-(2-hydroxyethyl)piperazine-N′-(2-hydroxypropancsulfonic acid) (HEPPSO) buffer, 2-(N-morpholine)ethanesulfonic acid (MES) buffer, triethanolamine buffer, imidazole buffer, glycine buffer, ethanolamine buffer, phosphate buffer, 3-(N-morpholine)-2-hydroxypropanesulfonic acid (MOPSO) buffer, piperazine-N,N′-bis(2-ethanesulfonic acid) (PIPES) buffer, piperazine-N,N′-bis(2-hydroxypropanesulfonic acid) (POPSO) buffer, N-tris[(hydroxymethyl)methyl]-3-aminopropanesulfonic acid (TAPS) buffer, 2-hydroxy-3-[tris(hydroxymethyl)methylamino]-1-propanesulfonic acid (TAPSO) buffer, N—[Tris(hydroxymethyl)methyl]-2-aminoethanesulfonic acid (TES) buffer, N-[Tris(hydroxymethyl)methyl]glycine (tricine) buffer, 2-amino-2-methyl-1,3-propanediol buffer, 2-amino-2-methyl-1-propanol buffer, and combinations thereof.

A composition (e.g., a cell and/or macromolecule stabilizing composition), in some embodiments, may include, without limitation, a surfactant and/or a reducing agent. A surfactant, in some embodiments, may include a detergent. A detergent may include, for example, an anionic detergent, a non-ionic detergent, and/or a cationic detergent. A nonionic detergent may include polyoxyethylene (20) sorbitan monolaurate, octyl-phenoxypolyethoxyethanols, nonyl-phenoxypolyethoxyethanols, octyl flucopyranosides, dodecyl maltopyranosides, heptyl thioglucopyranosides, big CHAP detergents, Genapol X-80, Pluronic detergents, polyoxyethylene esters of alkylphenols (e.g., Triton), and/or derivatives and analogues thereof.

According to some embodiments of the disclosure, a composition (e.g., a cell and/or macromolecule stabilizing composition) may include a long chain fatty acid, a long chain fatty ester, a long chain fatty alcohol, lithium, heparin, heparinase, butylhexylcitrate, and/or combinations thereof. Compositions according to some embodiments of the disclosure were tested in flow cytometry methods. A composition (e.g., a cell and/or macromolecule stabilizing composition), in some embodiments, may exclude heparin. For example, where the presence of heparin is undesirable (e.g., where it may adversely effect PCR) heparin may be omitted. In some cases, heparinase may even be included in an amount sufficient to remove heparin.

A cell and/or a macromolecule stabilizing composition, according to some embodiments, may be prepared and/or used as a solid, a liquid, or a gas (e.g., a vapor).

In some embodiments, it may be desirable to have a stabilizing composition useful for both whole cell assays (e.g., flow cytometry) and molecular assays (e.g., PCR, RT-PCR, histochemistry). According to some embodiments, a cell and/or a macromolecule stabilizing composition may include (a) a chelator (e.g., a chelator selected from ethylenediaminetetraacetic acid (EDTA), [ethylenebis(oxyethylenenitrilo)]tetraacetic acid (EGTA), 1,2-bis(2-aminophenoxy)ethane-N,N,N′,N′-tetraacetic acid (BAPTA), and salts thereof), (b) at least one chelator enhancing component (e.g., a chelator enhancing component selected from guanidine, lithium chloride, sodium salicylate, sodium perchlorate, and sodium thiocyanate), (c) a base (e.g., a base selected from the group consisting of a purine base and a pyrimidine base), (d) an anticoagulant (e.g., a sulfated glycosaminoglycan), and (c) a plasticizer (e.g., a citrated alcohol). For example, a cell and/or a macromolecule stabilizing composition may include a chelator, a chelator enhancing component, and a base as described herein. According to some embodiments, a cell and/or a macromolecule stabilizing composition may stabilize one or more cells (e.g., red blood cell, white blood cell) with little or no coagulation or clumping. Such stabilized cells may be suitable for analysis by flow cytometry. A base may be present at a concentration of from about 0.01 mg/L to about 1 mg/L, from about 0.01 mg/L to about 0.5 mg/L, and/or from about 0.2 mg/L to about 0.5 mg/L. A cell and/or a macromolecule stabilizing composition may further include plasticizer in some embodiments. A plasticizer may be present at a concentration of from about 0.1% (v/v) to about 10% (v/v), from about 0.2% (v/v) to about 5% (v/v), from about 0.5% (v/v) to about 2% (v/v), and/or from about 1% (v/v) to about 5% (v/v). A plasticizer may include a citrated alcohol in some embodiments. Examples of a citrated alcohol may include triethyl citrate, acetyl triethyl citrate, tributyl citrate, acetyl tributyl citrate, trioctyl citrate, acetyl trioctyl citrate, trihexyl citrate, acetyl trihexyl citrate, butyryl trihexyl citrate (e.g., n-butyryltri-n-hexyl citrate), trimethyl citrate, and combinations thereof.

A cell and/or a macromolecule stabilizing composition may include an anticoagulant, in some embodiments, at a concentration of from about 200 mg/L to about 20 g/L, from about 400 mg/L to about 5 g/L, from about 500 mg/L to about 2 g/L, and/or from about 1 g/L to about 3 g/L. An anticoagulant, in some embodiments, may include a sulfated glycosaminoglycan. Examples of a sulfated glycosaminoglycan may include, without limitation, heparin and/or a heparin salt (e.g., ammonium heparin, calcium heparin, lithium heparin, potassium heparin, sodium heparin, and/or zinc lithium heparin).

### Systems

A system, according to some embodiments of the disclosure, may include a cell and/or a macromolecule stabilizing composition and a sample storage container. For example, a system may include a container configured and arranged to receive a sample containing the macromolecule(s) and/or biomolecule(s) to be preserved and/or stabilized. A container may be configured and arranged to contact the sample with a cell and/or a macromolecule stabilizing composition. In a simple example, a cell and/or a macromolecule stabilizing composition formulated as a solid (e.g., tablet, powder, or hydrogel) may be deposited in the bottom of a small tube. Upon placing a sample (e.g., a liquid sample) in the tube, the cell and/or a macromolecule stabilizing composition may contact and mix with the sample milieu. The sample may be contacted (e.g., mixed) with a cell and/or a macromolecule stabilizing composition at the same time it is placed in a container or at some time thereafter.

In some embodiments of the disclosure, a system may include a cell and/or a macromolecule stabilizing composition further including a lipid, surfactant, and/or detergent. For example, a cell and/or a macromolecule stabilizing composition may be comprised in a micelle, a liposome, a vesicle, and/or a membrane-bound space.

A system, according to some embodiments, may include a cell and/or a macromolecule stabilizing composition and instructions for use. In some embodiments, a system may include a cell and/or a macromolecule stabilizing composition, a sample storage container, and instructions for use. A system may also include a shippable container configured to contain a sample storage container and its contents.

A system may include, according to some embodiments, an analytical device for analyzing a preserved molecule and/or cell. Examples of an analytical device may include, without limitation, a microscope, a plate-reader, a size-fractionating gel, a thermocycler, a flow cytometer, automated hematology analyzer, differential cell counter, cell sorter, beads (e.g., magnetic beads), an affinity matrix, and/or a spectrometer.

### Methods

A method of preserving and/or stabilizing a macromolecule and/or biomolecule (a “macromolecule stabilizing method”), according to some embodiments of the disclosure, may include contacting the macromolecule with a macromolecule stabilizing composition. For example, a bodily fluid comprising a macromolecule may be contacted with a macromolecule stabilizing composition having a chelator, a chelator enhancing component, and a purine base (e.g., adenine). A method of preserving and/or stabilizing a cell (e.g., a whole cell) (a “cell stabilizing method”), according to some embodiments of the disclosure, may include contacting the cell with a cell and/or macromolecule stabilizing composition. For example, a bodily fluid comprising a cell may be contacted with a cell and/or macromolecule stabilizing composition having a chelator, a chelator enhancing component, and a purine base (e.g., adenine).

The present disclosure also relates to methods for improving the signal response of a molecular assay of a test sample, including contacting the test sample with a cell and/or a macromolecule stabilizing composition to produce a preserved and/or stabilized test sample (“preserved test sample”), isolating and/or purifying a molecular analyte of interest from the test sample, and performing a molecular assay on the isolated and/or purified molecular analyte of interest. Without being limited to any particular mechanism of action, improved signal response in a nucleic acid assay may be due in part to enhanced hybridization as a result of the use of a cell and/or a macromolecule stabilizing composition of the present disclosure.

The present disclosure further relates to methods for improving hybridization of nucleic acids, including contacting a test nucleic acid with a cell and/or a macromolecule stabilizing composition to form a test solution and contacting the test solution with a target nucleic acid under conditions that permit test nucleic acid—target nucleic acid hybridization.

According to some embodiments, a method may comprise sufficiently stabilizing and/or preserving a cell such that the cell may be subjected to analysis by flow cytometry. For example, a method may include preserving and/or stabilizing a cell such that the cell (e.g., the milieu in which it is located) is free of clumps and/or debris that may interfere with flow analysis. Preservation and/or stabilization may be assessed using any available metric or combination of metrics. Formation of clumps and/or debris may be used as a preservation and/or stabilization metric. Appearance (e.g., color) may also be used as a preservation and/or stabilization metric.

A preservation and/or stabilization metric may include, for example, the presence of one or more markers (e.g., extracellular markers) over time. Preservation and/or stabilization markers may comprise one or more proteins, one or more carbohydrates, one or more lipids, one or more nucleic acids, and/or combinations thereof. For example, a preservation marker may include one or more lymphocyte surface markers. Examples of markers may include, for example, B-cell markers (e.g., CD19, CD20, CD21, CD22, and combinations thereof), T-cell markers (e.g., CD2, CD3, CD4, CD5, CD7, CD8, CD10, and combinations thereof), NK-cell markers (e.g., CD16, CD56, CD57, and combinations thereof), myeloid markers (e.g., CD13, CD33, CD34, and combinations thereof), monocyte markers (e.g., CD14), and/or pan leukocyte markers (e.g., CD45). In some embodiments, a cell (e.g., a lymphocyte) contacted with a composition (e.g., a cell and/or macromolecule stabilizing composition) may retain more than about 80%, more than about 90%, more than about 95%, and/or more than about 99% of one or more markers (e.g., cell surface markers), in terms of percentages and/or absolute counts, at about 48 hours, about 72 hours, and/or about 96 hours at room temperature (e.g., about 20° C.). For example, a lymphocyte contacted with a composition may retain at least about 80% of one or more T-cell markers (e.g., CD3, CD4, CD8) for about 96 hours (t₀ is the time the cell contacts the composition) at room temperature. Another example of a metric may be the quantity and/or quality of one or more nucleic acids detected in and/or recovered from a preserved and/or stabilized cell.

In some embodiments, the volume and/or weight ratio of cell and/or macromolecule stabilizing composition to sample may be from about 1:10 to about 10:1, from about 1:10 to about 1:1, and/or from about 1:10 to about 1:5. A cell and/or macromolecule stabilizing composition may be combined with a sample at a ratio of from about 10 μg to about 10 mg of cell and/or macromolecule stabilizing composition per milliliter and/or gram of sample. A cell and/or macromolecule stabilizing composition may be added to a sample to be preserved and/or stabilized (e.g., a vessel containing the sample) according to some embodiments. A sample to be preserved and/or stabilized may be added, in some embodiments, to a cell and/or macromolecule stabilizing composition (e.g., a vessel containing the cell and/or macromolecule stabilizing composition). According to some embodiments, a cell and/or macromolecule stabilizing composition and a sample to be preserved and/or stabilized may be added to each other at the same time. For example, both may be added to an otherwise empty mixing vessel.

As will be understood by those skilled in the art, other equivalent or alternative compositions, systems, and methods for preserving and/or stabilizing a cell and/or a macromolecule and/or biomolecule according to embodiments of the present disclosure can be envisioned without departing from the essential characteristics thereof. For example, a cell and/or a macromolecule stabilizing composition may be formulated as a powder, granule, tablet, capsule, liquid, syrup, paste. A cell and/or a macromolecule stabilizing composition may be deposited in a sample container by any available method. For example, a cell and/or a macromolecule stabilizing composition may be coated (e.g., sprayed or spray-dried) onto an inner surface of a sample container before a macromolecule-containing sample is introduced. A cell and/or a macromolecule stabilizing composition may also be simply placed in a sample container in a solid or liquid form. Alternatively, a cell and/or a macromolecule stabilizing composition may be kept in a separate container and only contacted with a sample after the sample has been placed in a sample container. Also, where ranges have been provided, the disclosed endpoints may be treated as exact and/or estimates as desired or demanded by the particular embodiment In addition, it may be desirable in some embodiments to mix and match range endpoints. In some embodiments, the term “about” when applied to a numeric value may refer to that numeric value plus or minus about 1% of that value, plus or minus about 5% of that value, plus or minus about 10% of that value, plus or minus about 25% of that value, and/or plus or minus about 50% of that value. When the numeric value is provided as an endpoint to a range, the term “about” may have more or less flexibility depending on the extent of the range, according to some embodiments. For example, if the range covers a single order of magnitude (e.g., from about 1 to about 10), “about” may have less flexibility. For a range that covers several orders of magnitude (e.g., from about 0.1 to about 100), however, the endpoints may have more flexibility. In some embodiments, a concentration range that includes the term “up to” (e.g., up to 1 mM of NaCl) may include a lower endpoint that reaches any amount of the material above zero (e.g., any trace of NaCl). The term “up to,” in some embodiments, may contemplate and/or require that some non-zero amount of the specified material is present. These equivalents and alternatives along with obvious changes and modifications are intended to be included within the scope of the present disclosure. The present disclosure is intended to be illustrative, but not limiting, of the scope of the disclosure. The appended claims are similarly intended to be illustrative, but not limiting, of the scope of the disclosure.

Some specific embodiments of the disclosure may be understood, by referring, at least in part, to the following specific example embodiments. These examples illustrate some, but not necessarily all, aspects of some embodiments of the disclosure and additional variations will be apparent to one skilled in the art having the benefit of the present disclosure.

### EXAMPLE 1

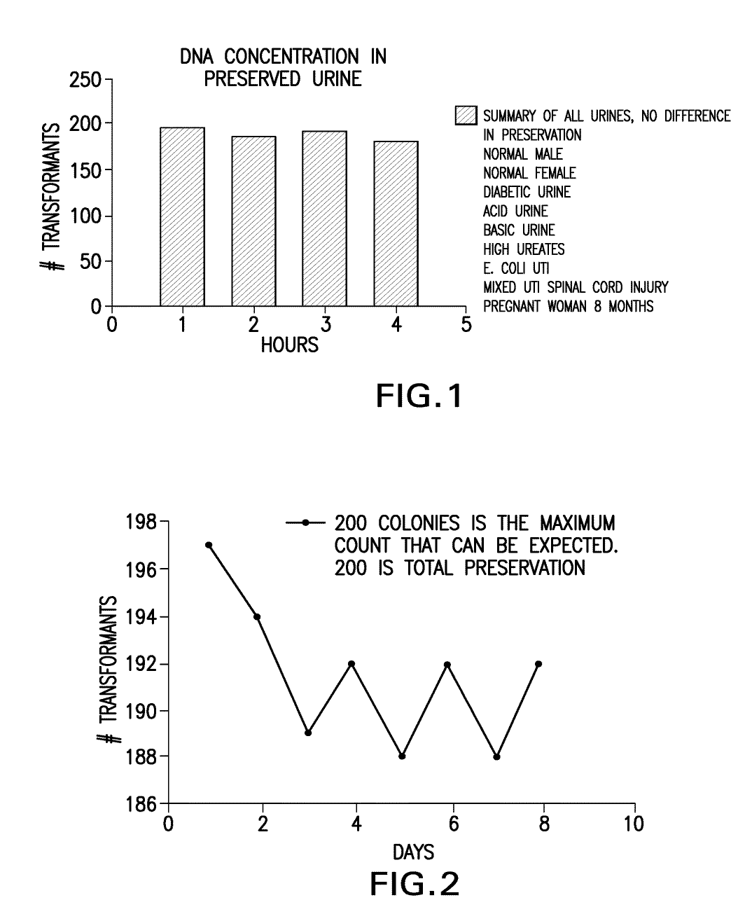

FIG. 1 is a bar graph of DNA concentration in urine preserved and/or stabilized in accordance with an embodiment of the disclosure. The number of transformants in ten types of urine specimens were tested using a GTT, counted hourly, and then summarized. The standard Gonostat protocol (see Example 2, infra) was employed, and the preservative used was 1M guanidine HCl/0.01M EDTA. A count of two hundred colonies demonstrates total preservation of a specimen. The number of gonococcal transformants in the preserved urine remained relatively constant approaching two hundred, throughout the four hours of the test. No significant difference in level of preservation was observed among the different types of urine specimens. Therefore, the example composition tested provided nearly total protection for DNA in urine.

### EXAMPLE 2

FIG. 2 is a graph of eight day GTT serial data on urine preserved and/or stabilized in accordance with an embodiment of the disclosure. 1 pg of gonococcal DNA was spiked into 9 mL of fresh human urine and 1 mL of aqueous a macromolecule stabilizing composition containing 1M sodium perchlorate and 0.01M EGTA. 300 μL was spotted onto a lawn of the Gonostat organism at 24 hour intervals for eight days. The plates contained BBL Chocolate 11 agar and were incubated at 37° C. for 24 hours before readings were taken. The number of colonies observed throughout the eight-day testing period ranged from a low count of one hundred eighty-eight to a high count of one hundred ninety-seven. Thus, embodiments of the disclosure may preserve and/or stabilize DNA in urine for a significantly longer period of time than previously provided.

### EXAMPLE 3

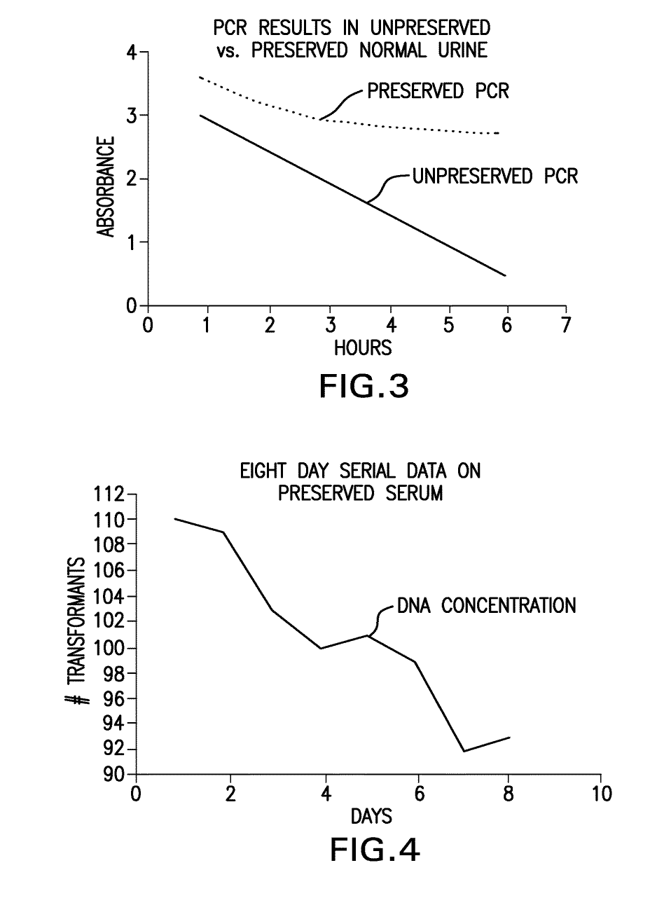

FIG. 3 is a graph comparing PCR results in unpreserved and preserved (preserved and/or stabilized) normal urine according to an embodiment of the disclosure. A MOMP template to Chlamydia trachomatis was used and amplified using a standard PCR protocol. 200 copies of the MOMP target were spiked into 9 mL of fresh human urine containing 1M sodium perchlorate and 0.01M BAPTA. PCR was done each hour for eight hours total. In the unprotected urine, approximately three PCR absorbances were measured one hour after the addition of DNA to the urine. The number of PCR absorbances approached zero by the sixth hour. By contrast, in the preserved and/or stabilized specimen, in excess of three PCR absorbances were measured at the one hour testing. However, approximately three PCR absorbances were still observed by the sixth hour. Therefore, embodiments of the disclosure may preserve and/or stabilize sufficient DNA and nucleic acid sequences to permit PCR testing well beyond the testing limits of unpreserved urine. The results shown in the Figure are consistent for all types of DNA in a urine specimen.

### EXAMPLE 4

The reagents and methods of the disclosure may be used for preserving other bodily fluids and excretions, such as blood serum. FIG. 4 is a graph of eight day serial data on preserved and/or stabilized serum according to an embodiment of the disclosure. The protocol used was similar to Example 3, except fresh human serum was used. The number of transformant colonies observed throughout the eight-day testing period ranged from a high count of one hundred ten at the one day measurement to a low count of approximately ninety-two at the seven day measurement. In fact, the test results actually showed an increase in transformant colonies between days seven and eight. Thus, some embodiments of the disclosure preserve and/or stabilize DNA in serum for a significantly longer period of time than previously attainable.

### EXAMPLE 5

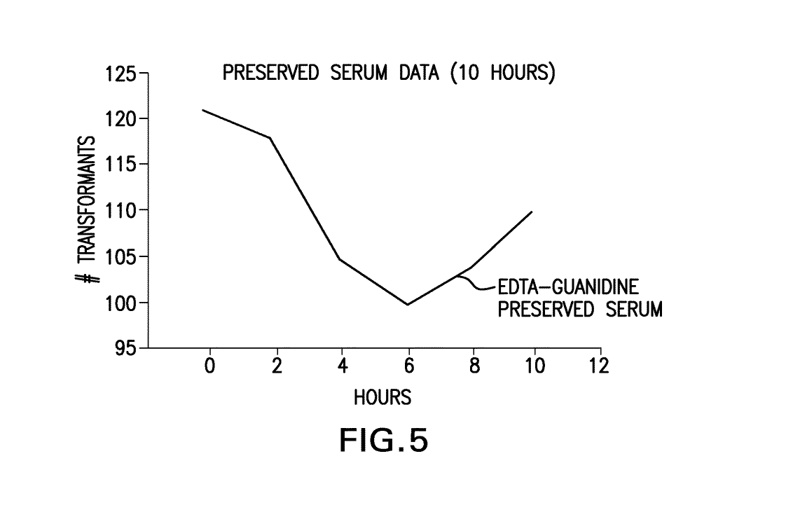

FIG. 5 is a graph of DNA concentration in preserved and/or stabilized serum according to an embodiment of the disclosure. The scrum was preserved and/or stabilized with a macromolecule stabilizing composition comprising 1M guanidine HCl/0.01M EDTA. The protocol used was similar to Example 3, except fresh human serum was used, and the duration time of the study was ten hours. In excess of 120 transformants were measured at the time gonococcal DNA was added to the serum. Approximately 100 transformants were counted at the six hour measurement. However, by the tenth hour, testing indicated that the concentration of biologically active DNA in the preserved serum had increased to approximately 110 transformant colonies.

### EXAMPLE 6

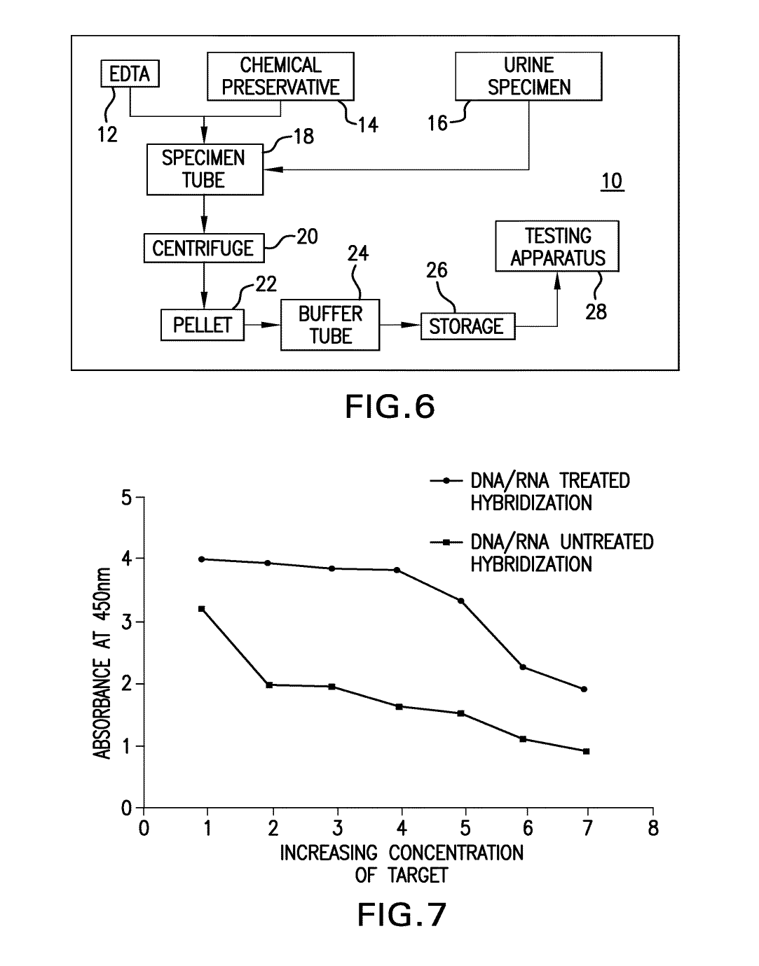

An example embodiment of a method 10 for preserving DNA is illustrated diagrammatically in FIG. 6. This protocol is described in Table 1, below and has been observed to produce high yields of DNA/RNA suitable for such testing methods as PCR, restriction fragment length polymorphisms assay (RFLP), and nucleic acid probes using urine specimens.

TABLE 1

| | |
|---|---|
| 1. | 10 ml of clean catch urine 16 is added to a specimen test tube 18 |
| | containing divalent metal chelator 12 and chelator enhancing |
| | component 14. Test tube is inverted two or three times to mix the |
| | urine. |
| 2. | Test tube is transported to laboratory. No refrigeration is necessary. |
| | Note: The test tube should be stored in a cool place and not in direct |
| | sunlight. |
| 3. | At the laboratory, the test tube is centrifuged 20 at 3200 rpm for 10 |
| | minutes. |
| 4. | Using a sterile transfer pipette, the pellet 22 at the bottom of the |
| | test tube is transferred to another test tube containing buffer 24. |
| | (As little urine as possible should be transferred with the pellet |
| | material.) |
| 5. | The buffered material is stored 26 at between 2-8° C. until |
| | ready to test 28. |
| 6. | The specimen size necessary to run the assay needs to be validated |
| | on the individual test methodology and individual testing protocol |
| | being used. |

### EXAMPLE 7

### Preservation of DNA in Simulated Clinical Specimens

In the following experiment, simulated clinical urine specimens were produced and tested for the presence of gonococcal DNA. The chemicals listed in Table 2, below, were added, at the concentrations previously described, to urine specimens from healthy adults, as was EDTA.

A suspension of gonococci was immediately added to each urine specimen. The added gonococci were an ordinary strain of N. gonorrhoeae, 49191, which was grown overnight on GC agar medium at 37° C. in a 5% CO₂ atmosphere. The N. Gonorrhoeae colonies were picked and suspended in GC buffer. A 1/10 volume of a suspension containing approximately 10 Colony forming units (cfu) per mL was added to the urine. As a positive control, the suspension of gonococci was also added to Hepes buffer.

All simulated clinical specimens and the Hepes controls were tested at time zero, i.e., when the chemicals and gonococci were added. The specimens and controls were also tested after storage at room temperature for six days. This six day period was selected to approximate the maximum time expected between collecting, mailing, and testing patient specimens.

With the exception of urine samples containing Sodium dodecyl sulfate (SDS) and sarkosyl, the simulated specimens and Hepes controls were processed as follows:

1. A 10 mL quantity was centrifuged at 4000 rpm for 30 minutes.

2. The supernatant was decanted, and the pellet was suspended in 1 mL phosphate buffer.

3. The suspension was heated for 10 minutes in a water bath at 60° C.

4. After cooling, the suspension was used in the GTT.

The simulated urine specimens containing SDS-EDTA or sarkosyl-EDTA were processed as follows:

1. Approximately a 2½ volume (approximately 25 mL) of 95% ethyl alcohol was added to the tube with the urine and macromolecule stabilizing composition. The contents were mixed by inverting the tube several times.

2. The mixture was centrifuged at 4000 rpm for 30 minutes.

3. The pellet was suspended in 10 mL of 70% alcohol and centrifuged.

4. The pellet was then suspended in 1 mL phosphate buffer.

5. The suspension was heated for 10 minutes in a water bath at 60° C.

6. After cooling, the suspension was used in the GTT.

The inoculated urine was stored at room temperature for 6 days prior to testing. The formulations that preserved and/or stabilized (+) or did not preserve and/or stabilize (−) gonococcal DNA in the inoculated urine for six days to approximately the same degree as in the Hepes buffer control are indicated. Although the results of the Gonostat™ assay may be semi-quantitated, the tests were not designed to rank the relative efficacy of the macromolecule stabilizing compositions. Thus, the results given in Table 2 indicate whether or not the particular chemical preserved and/or stabilized DNA in urine over a six day period to same degree as in the Hepes buffer.

TABLE 2
Macromolecule Stabilizing Composition

| + | − |
|---|---|
| 0.01M EDTA + Guanidine hydrochloride (1M) | Sodium |
| 0.01M EDTA + Guanidine thiocyanate (1M) | periodate (1M) |
| 0.01M EDTA + Lithium chloride (1M) | Trichloroacetic |
| 0.01M EDTA + Manganese chloride (1M) | acid (1M) |
| 0.01M EDTA + Sarkosyl (1%) | Urea (1M) |
| 0.01M EDTA + Sodium dodecyl sulfate (SDS) (1%) | |
| 0.01M EDTA + Sodium perchlorate (1M) | |
| 0.01M EDTA + Sodium salicylate (1M) | |
| 0.01M EDTA + Sodium thiocyanate (1M) | |

The 92% sensitivity exhibited with male urine specimens is comparable to the culture results reported in the literature. In addition, the 88% sensitivity exhibited with female urine specimens exceeds the previously-reported levels.

While a preferred embodiment of the disclosure is directed to the preservation of gonococcal DNA, it will be readily apparent to one skilled in the art that the disclosure is adaptable for use in preserving other types of DNA, such as that of Haemophilus influenzae and Bacillus subtilis. Some embodiments of the disclosure may also be used to preserve and/or stabilize RNA contained in bodily fluid samples. Such preserved RNA may be used for RNA transcriptase and reverse transcriptase assays for viral segments and human gene sequence testing.

Furthermore, although a macromolecule stabilizing composition may be added to a bodily fluid, e.g., a urine specimen, a urine specimen may also be added to a macromolecule stabilizing composition without detriment to the efficacy of preservation/stabilization. Optimal preservation of the DNA may be achieved by adding a single macromolecule stabilizing composition of the disclosure to a specimen.

### EXAMPLE 8

### PCR Detection of Penicillinase-Producing Neisseria gonorrhea

The PCR signal-enhancing effect of a macromolecule stabilizing composition of the disclosure is demonstrated by the following example. Four varieties of TEM-encoding plasmids are found in PPNG. These are the 6.7 kb (4.4 Mda) Asian type, the 5.1 kb (3.2 Mda) African type, the 4.9 kb (3.05-Mda) Toronto type and the 4.8 kb (2.9-Mda) Rio Type. This PCR assay for PPNG takes advantage of the fact that the TEM-1 gene is located close to the end of the transposon Tn2; by the use of one primer in the TEM-1 gene and the other in a sequence beyond the end of Tn2, and common to all four plasmids, a PCR product only from plasmids and not from TEM-1 encoding plasmids was obtained. (Table 3, below) The conditions associated with this protocol were modified to include the macromolecule stabilizing composition in the hybridization and the treated probe was mixed with the 761-bp amplification product per standard PCR protocol. The results were read at A₄₅₀ nm.

### Materials and Reagents

BBL chocolate 11 agar plates

Sterile Tris Buffer 10 mM Tris (pH 7.4), 1 mM EDTA

0.5-mL Gene Amp reaction tubes

Sterile disposable Pasteur pipette tips

Aerosol-resistant tips

PCR master mix:
50 mM KCl
2 mM MgCl
50 μM each of
   Deoxyribonucleoside triphosphate;
   2.5 U of Taq Polymerase (Perkin Elmer);
   5% glycerol;
   50 pmol each of primers PPNG-L and PNG-R (per 100 μL reaction)

### Denaturation Solution

1M Na 5×Denhardt's solution

### Prehybridization Solution

5×SSC(1×SSC is 0.015 M NaCl plus 0.015 M sodium citrate);

5×Denhardt's solution;

0.05% SDS;

0.1% Sodium Ppi, and

100 μg of sonicated salmon sperm DNA per mL.

### Hybridization Solution

Same as prehybridization solution but without Denhardt's solution and including 200 μL of macromolecule stabilizing composition 1.
   1 mL DNA/RNA macromolecule stabilizing composition (1M guanidine HCl/0.01M EDTA)
   Avidin-HRP peroxidase complex (Zymed)
   Magnetic microparticles (Seradyne)

TABLE 3

| Function | Name | Nucleotide sequence 5′ to 3′ |
|---|---|---|
| Primer | PPNG-L | AGT TAT CTA CAC GAC GG |
| | | (SEQ ID NO: 1) |
| Primer | PPNG-R | GGC GTA CTA TTC ACT CT |
| | | (SEQ ID NO: 2) |
| Probe | PPNG-C | GCG TCA GAC CCC TAT CTA |
| | | TAA ACT C (SEQ ID NO: 3) |

### Methods

Sample preparation: 2 colonies were picked from a chocolate agar plate. Colonies were suspended in deionized water just prior to setting up PCR. The master mix was prepared according to the recipe above. 5 μL of the freshly prepared bacterial suspension was added to 95 μL of master mix. The DNA was liberated and denatured in a thermocycler using three cycles of 3 min at 94° C. and 3 min at 55° C. The DNA was amplified in the thermal cycler by using a two step profile: a 25 s denaturation at 95° C. and a 25 s annealing at 55° C. for a total of thirty cycles. The time was set between the two temperature plateaus to enable the fastest possible annealing between the two temperatures. 15 pmol of labeled (avidin-HRP complex) detection probe PPNG-C was added to the hybridization solution bound to magnetic micro particles with and without the macromolecule stabilizing composition at 37° C. for 1 hour. The control and treated probes were then added to the amplification product and the reaction was colorimetrically detected at A₄₅₀ nm. The signal obtained from the hybridization probes treated with a macromolecule stabilizing composition of the disclosure was found to be significantly higher than the untreated probes.

### EXAMPLE 9

Compositions, systems, and methods in accordance with some embodiments of the disclosure may increase the signal obtained with a nucleic acid testing method, such as a polymerase chain reaction (PCR), LCₓ, and genetic transformation testing (GTT). For example, compositions, systems, and methods may enhance hybridization in such nucleic acid testing methods as the PCR. FIG. 7 illustrates the improvement in hybridization obtained a specific example embodiment of a macromolecule stabilizing composition disclosed herein on the hybridization of penicillinase-producing Neisseria gonorrhea (PPNG) DNA and PPNG-C probe. The PCR protocol was the same as described in Example 10.

### EXAMPLE 10

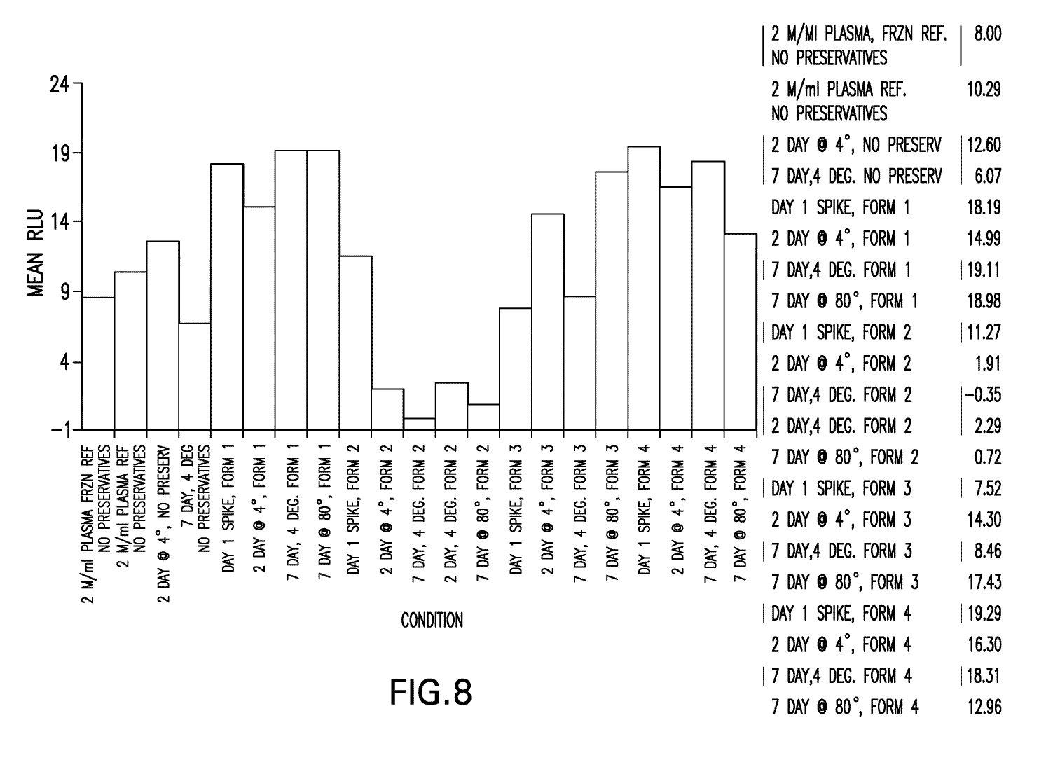

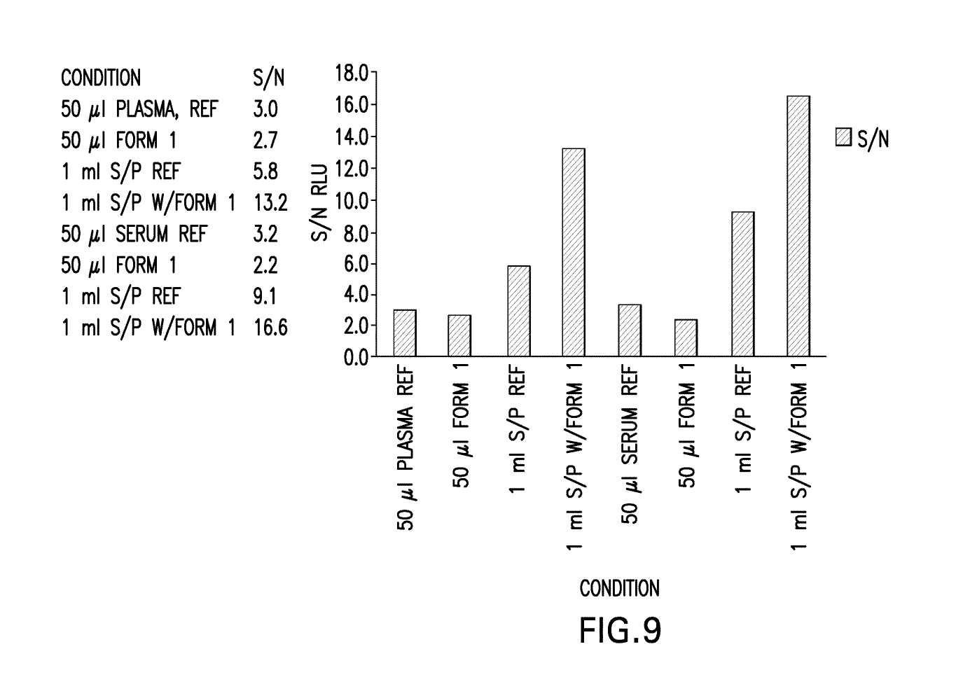

FIG. 8 and FIG. 9 further illustrate the efficacy of specific example embodiments of compositions, systems, and methods of the disclosure in improving the results obtained with nucleic acid testing methods, in this case, a branched DNA assay (Chiron). In the tests run in FIG. 8, a bDNA assay was used to assess the protective effect of the macromolecule stabilizing compositions. DNA sequences from the hepatitis C virus were spiked into serum and plasma. The protected serum and plasma were mixed with 9 mL of serum or plasma and 1 mL of macromolecule stabilizing composition. The following formulations were used: 1) 1M guanidine HCl/0.01M EDTA, 2) 1M sodium perchlorate/0.01M BAPTA, 3) 1M sodium thiocyanate/0.01M EGTA, and 4) 1M lithium chloride/0.01M EGTA. The formulations were stored for seven days at 4° C. bDNA assay relies on hybridization; it can be seen from clearly the absorbance results that the target sequences were not only protected against degradation, but the more than doubling of the absorbance results indicates an enhancement of hybridization/annealing of the target sequences.

FIG. 9 illustrates a serum v. plasma study. 50 μL samples of fresh human plasma, and 1 mL samples of fresh human serum were protected with 1M guanidine HCl/0.01M EDTA and the bDNA assay was run on these samples after the samples were stored at 20° F. for 48 hours. Results were compared to unprotected samples. It can be seen clearly from the absorbance results that the target sequences were not only protected against degradation, but the more than doubling of the absorbance results indicates an enhancement of hybridization/annealing of the target sequences.

### EXAMPLE 11

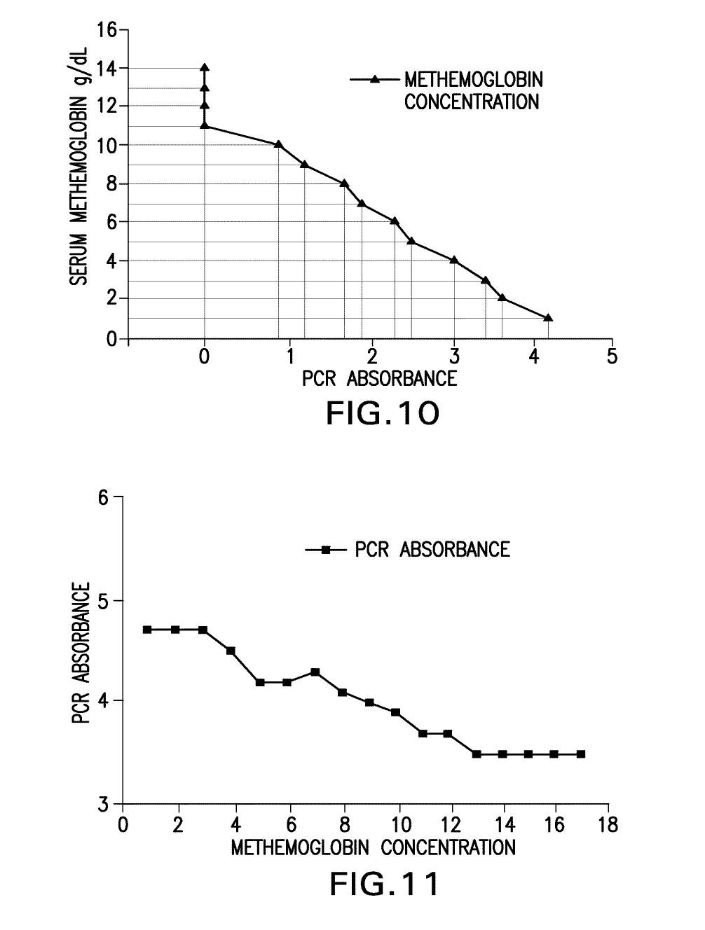

Heme compounds such as methemoglobin have been observed to interfere with PCR amplification of nucleic acids. For example, FIG. 10 shows the results of a series of PCR assays performed according to Example 10, wherein the template, fresh human serum, was spiked with increasing amounts of methemoglobin. As shown, the absorbance decreases as a function of methemoglobin concentration. At the highest concentrations, no absorbance (i.e., amplification) was observed at all.

Macromolecule stabilizing compositions of the disclosure, according to some embodiments, may remove the interference with heme compounds, e.g., methemoglobin, on PCR assays run on blood scrum. FIG. 11 illustrates the improvement (i.e., increased amplification as measured by absorbance (A₄₅₀)) obtained by adding to the serum sample a macromolecule stabilizing composition comprising 1 M sodium thiocyanate and 0.1 M EDTA. Like the control (FIG. 10), serum samples were spiked with increasing amounts of methemoglobin, to a concentration of 10 dl/mL. Serial PCR assays were run over a four hour period.

### EXAMPLE 12

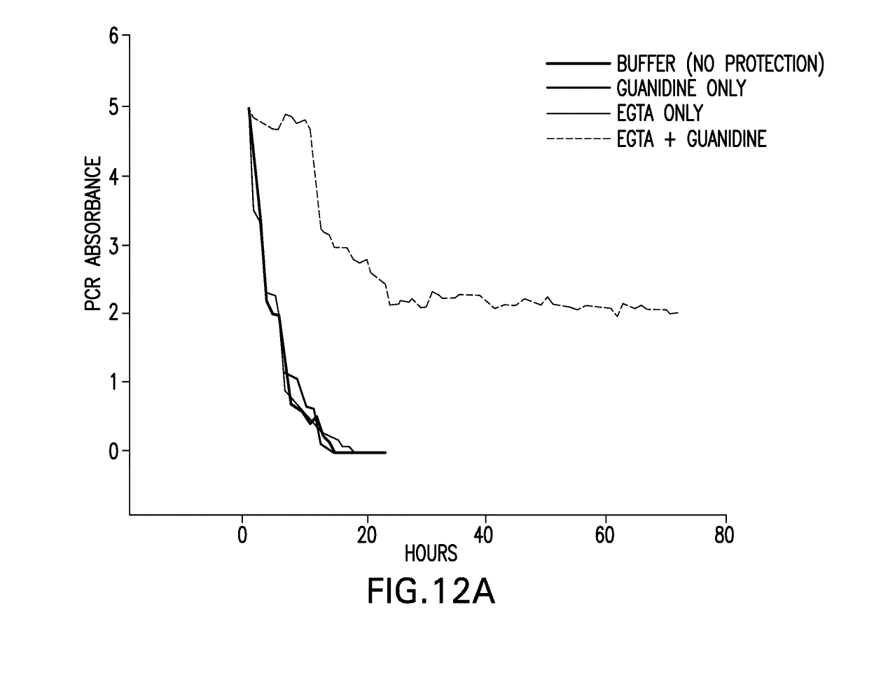

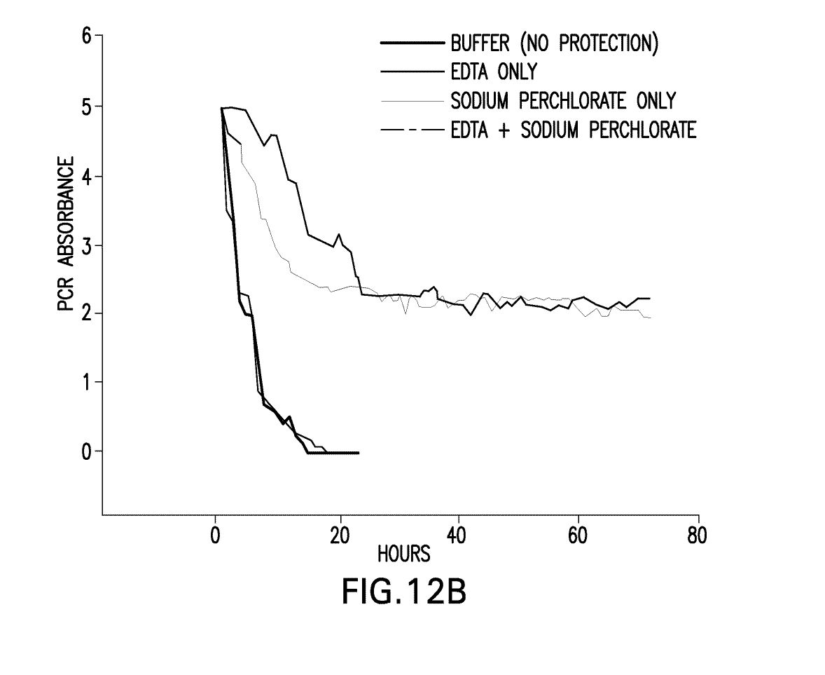

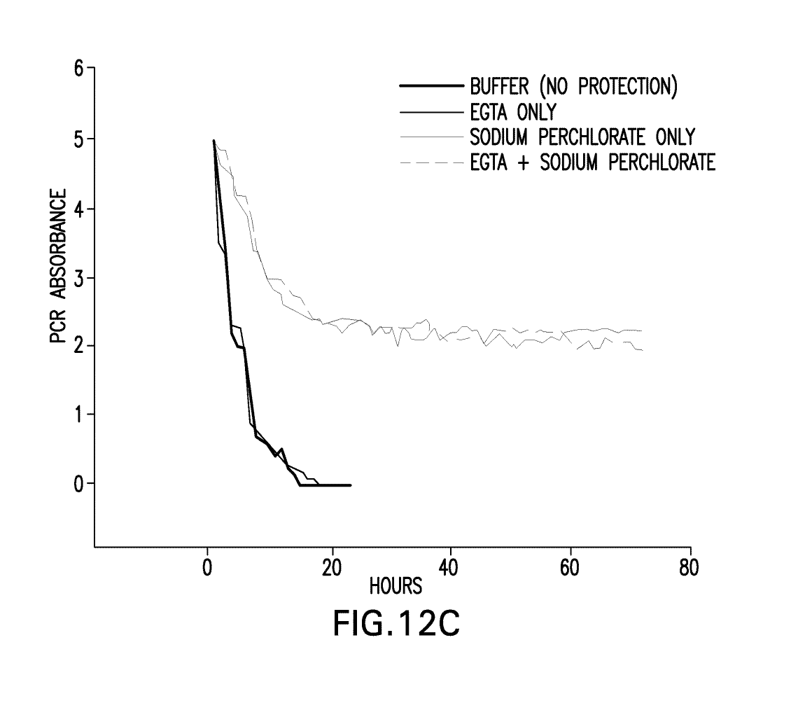

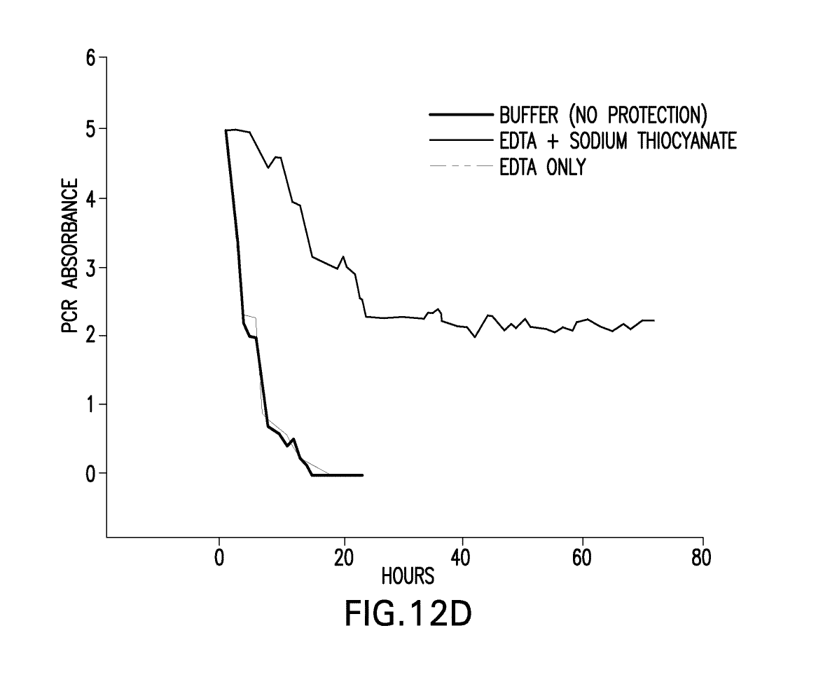

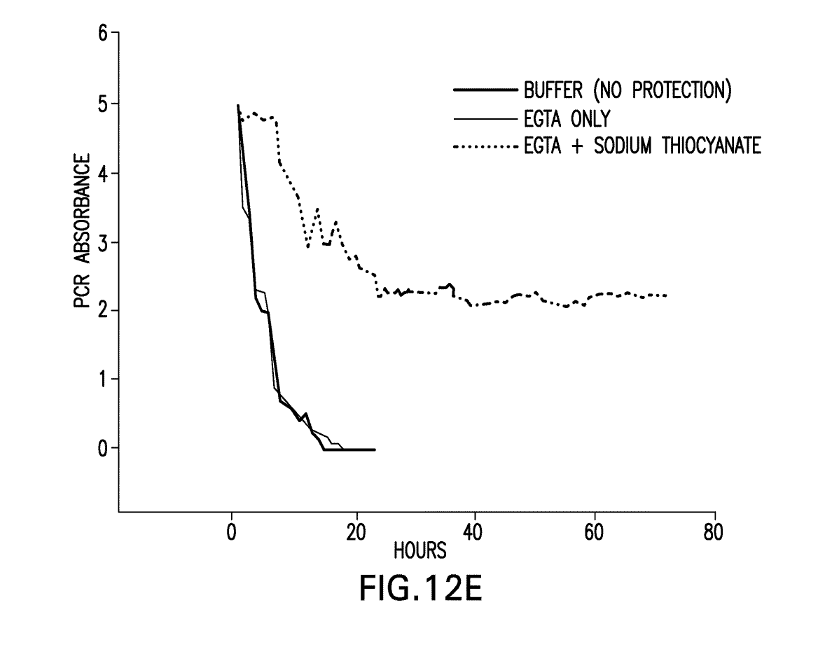

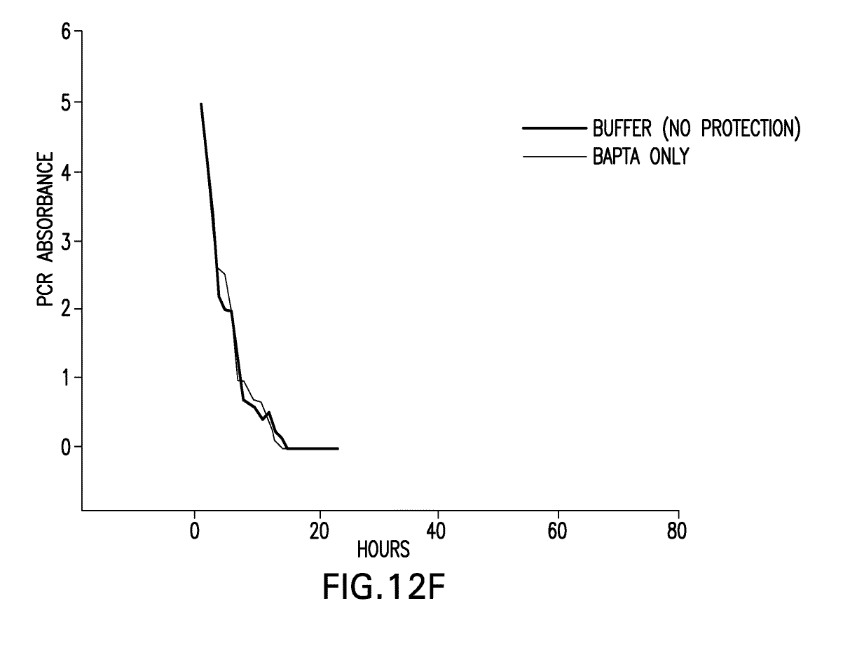

An example composition including a divalent metal chelator and a chelator enhancing component had a surprising and synergistic effect on protecting hepatitis B sequences in serum. Specifically, a hepatitis B template was contacted with a test composition (e.g., 1M sodium perchlorate/0.01 M EGTA) at room temperature for up to 36 hours (sampled at 2 hour intervals). Samples were subjected to PCR amplification using MD03 and MD06 primers using the sample PCR protocol as described in Example 10. A representation of the results obtained is provided in FIGS. 12A-12F. Collectively, these figures show that preservation and/or amplification of hepatitis B sequences is increased when specific example embodiments of macromolecule stabilizing compositions of the present disclosure are used compared to the addition of EGTA or sodium perchlorate individually.

### EXAMPLE 13

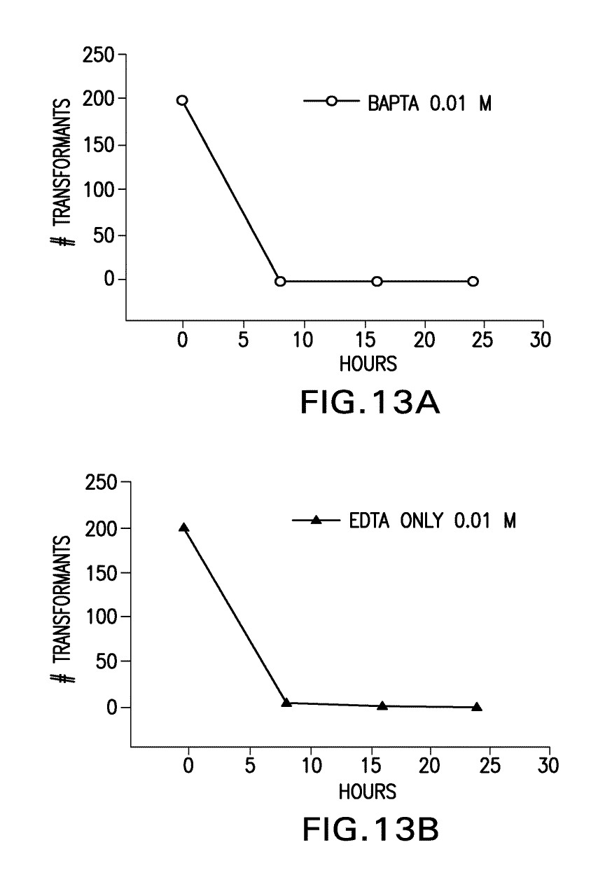

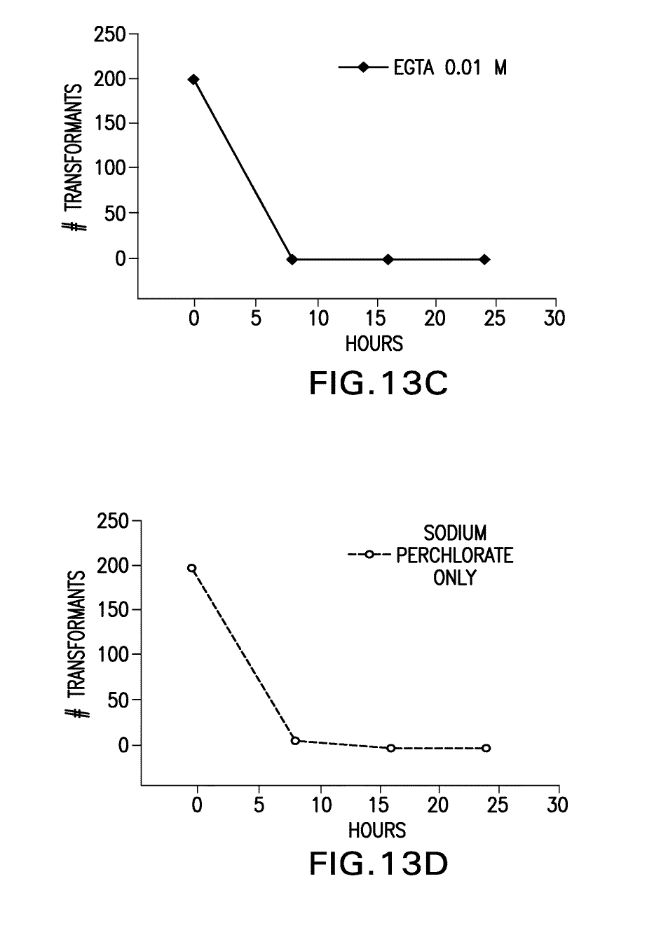

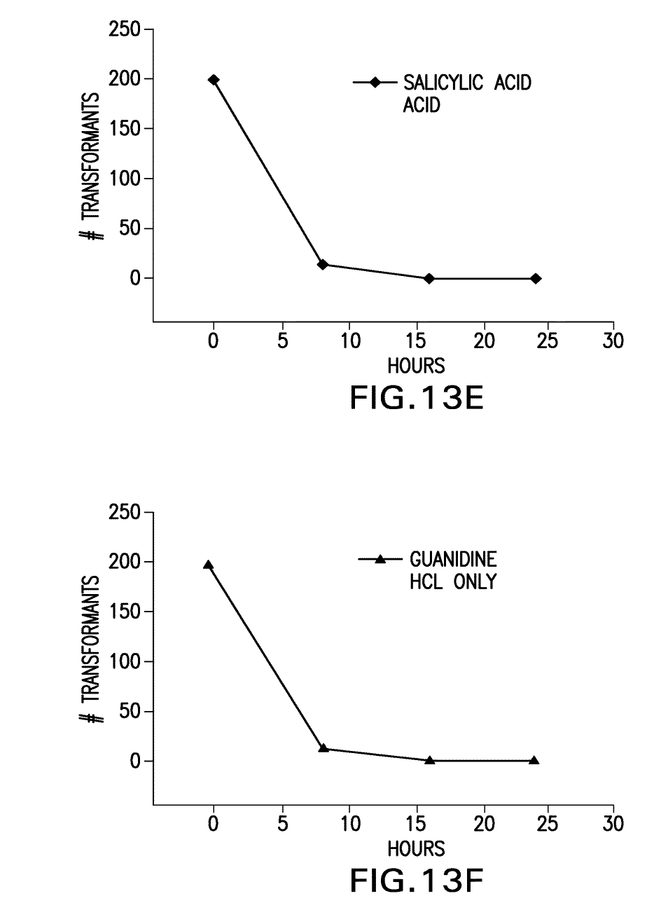

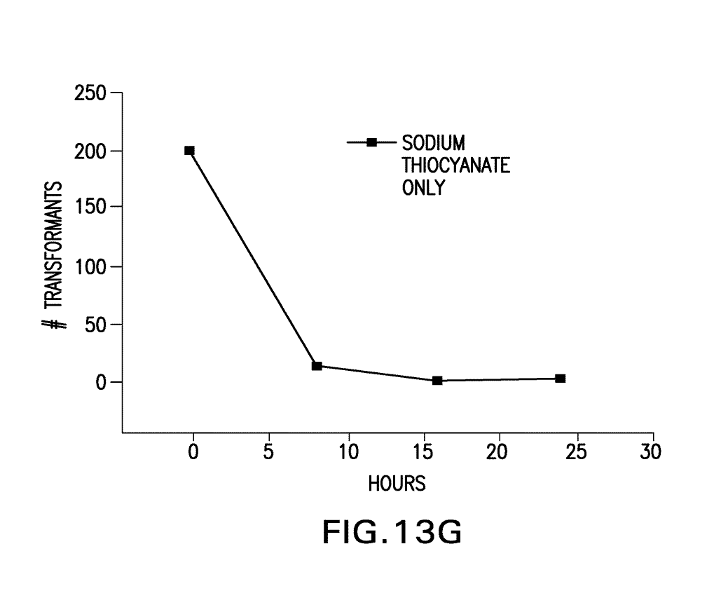

FIG. 13 illustrates a (relatively modest) preservative effect on gonococcal DNA in urine stored at room temperature and subsequently subjected to PCR detection provided by the individual addition of components of the reagents of the present disclosure, i.e., divalent metal chelators 0.01M BAPTA (FIG. 13A), 0.01M EDTA (FIG. 13B), 0.01M EGTA (FIG. 13C); and chelator enhancing components 1M sodium perchlorate (FIG. 13D), 1M salicylic acid (FIG. 13E), 1M guanidine HCl (FIG. 13F), 1M sodium thiocyanate (FIG. 13G), and lithium chloride (FIG. 13H). The number of transformants in ten types of urine specimens were tested using a GTT, counted hourly, and then summarized. A standard Gonostat protocol (see Example 2, infra) was employed and illustrated a synergistic effect obtained by the combination of divalent metal chelators and chelator enhancing components in protecting gonococcal DNA in urine stored at room temperature and subsequently subjected to PCR detection.

### EXAMPLE 14

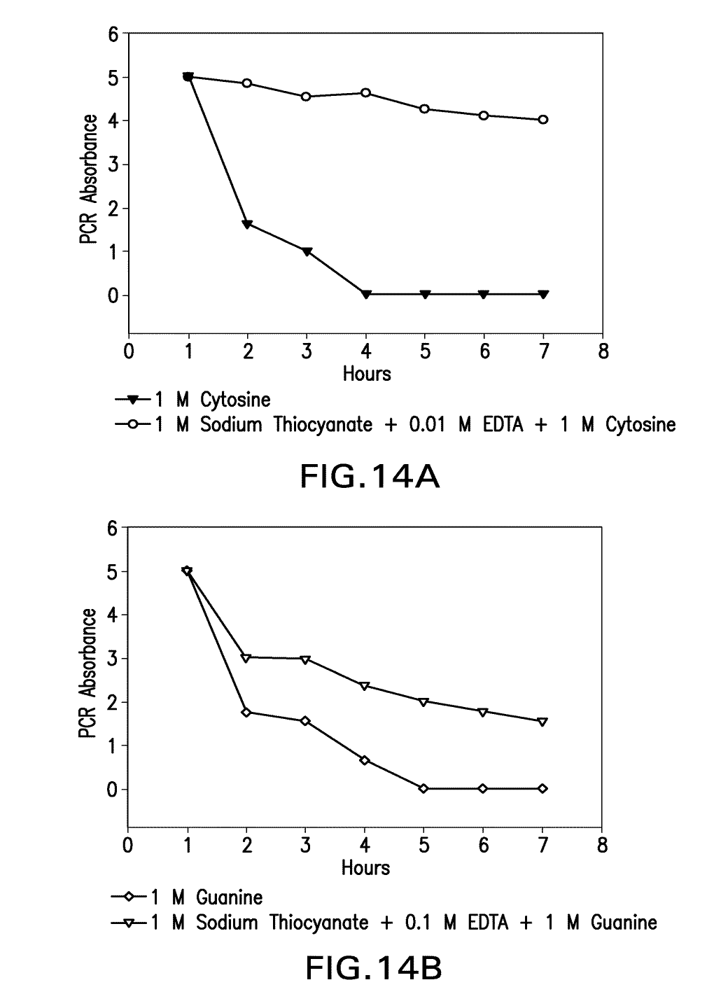

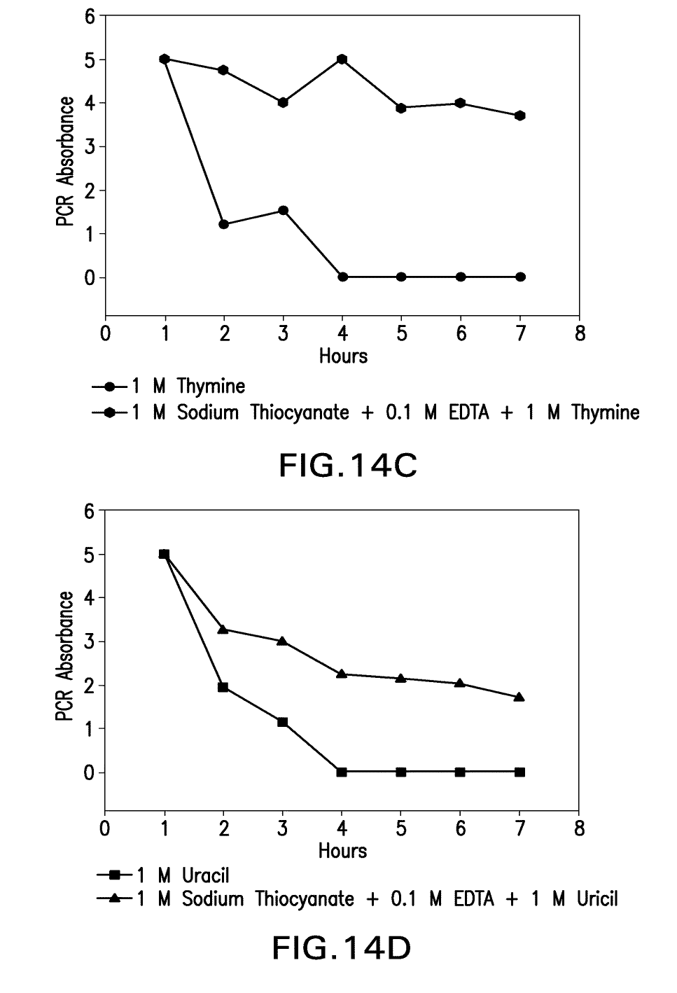

Compositions comprising purine bases or pyrimidine bases (1 M) were prepared either with or without sodium thiocyanate (1 M) and EDTA (0.1 M). Fresh samples of human urine were collected, spiked with 1 pg of gonococcal DNA, combined with one of the recited compositions, and incubated at room temperature. Aliquots were removed after 8 hours and tested by PCR for the presence of amplifiable gonococcal DNA. The PCR protocol was the same as described in Example 10. As illustrated in FIG. 14, compositions with sodium thiocyanate, EDTA, and a purine or pyrimidine base stabilized gonococcal DNA in urine more effectively than compositions with a purine or pyrimidine base alone.

### EXAMPLE 15

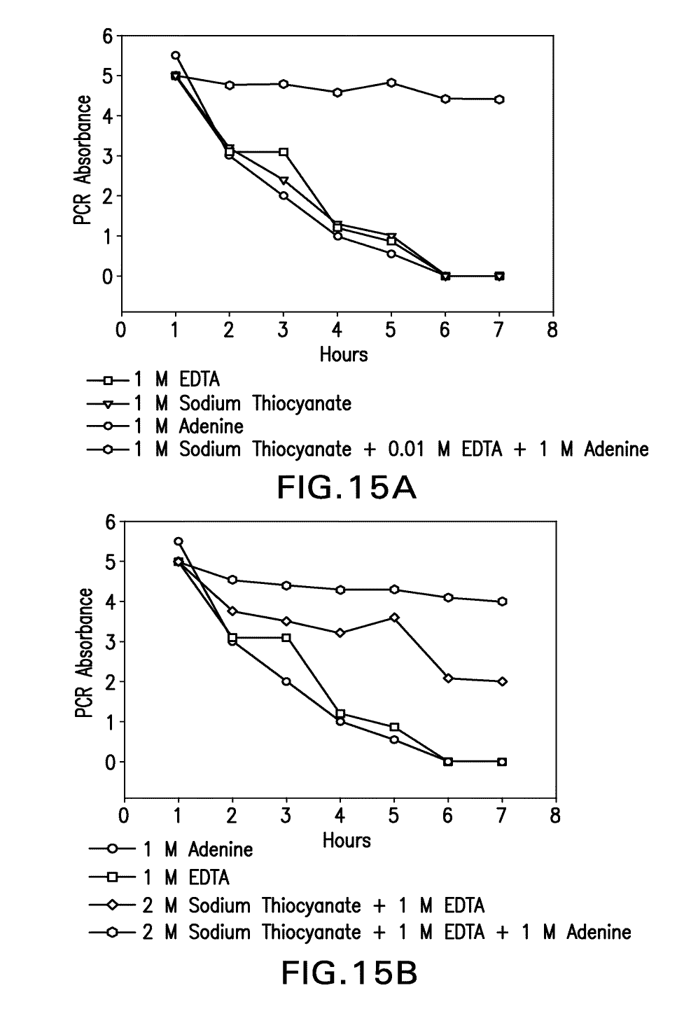

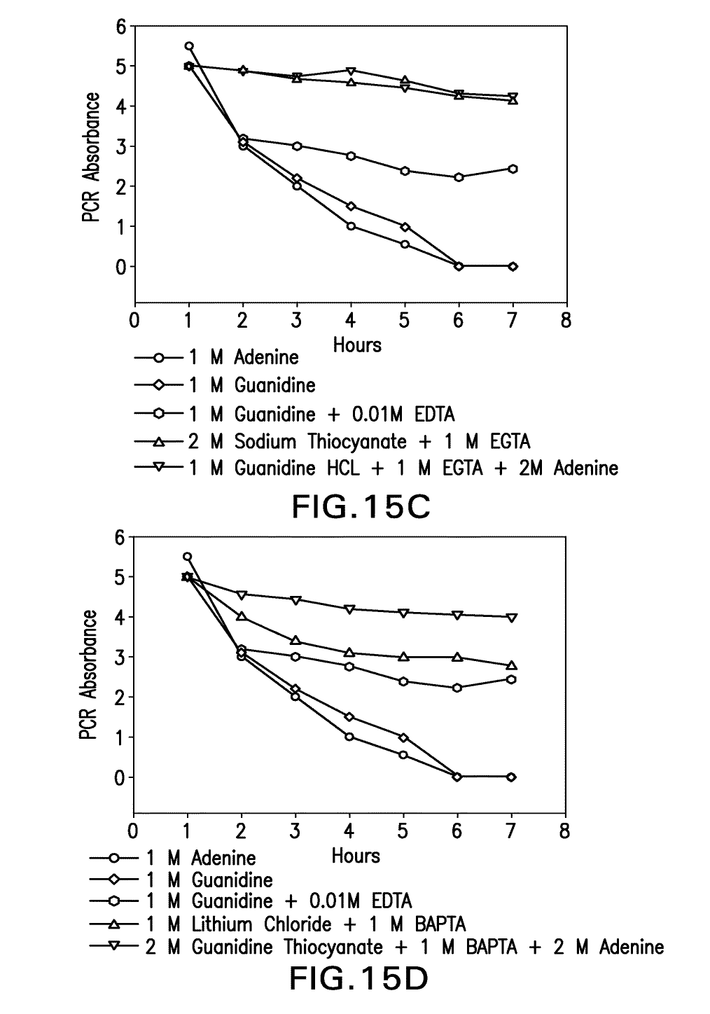

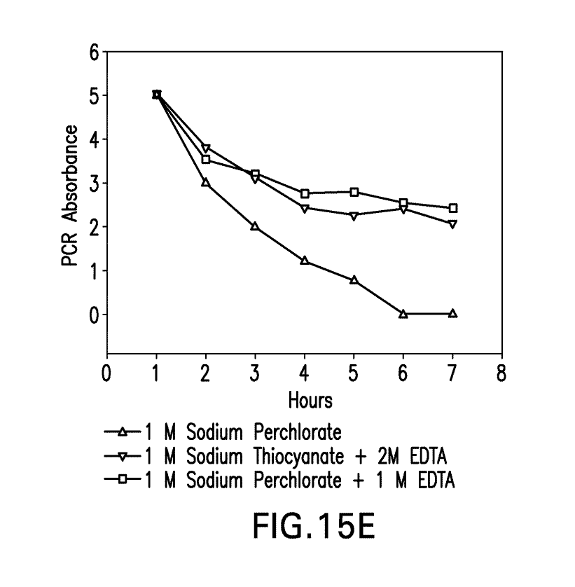

Compositions comprising sodium thiocyanate, EDTA, and/or adenine were prepared. Fresh samples of human urine were collected, spiked with 1 pg of gonococcal DNA, combined with one of the recited compositions, and incubated at room temperature. Aliquots were removed after 8 hours and tested by PCR for the presence of amplifiable gonococcal DNA. The PCR protocol was the same as described in Example 10. As illustrated in FIG. 15A, compositions with sodium thiocyanate, EDTA, and adenine generally stabilized gonococcal DNA in urine more effectively than compositions with fewer than all three components. The only exception observed was where the composition comprised sodium thiocyanate and EGTA.

### EXAMPLE 16

Compositions comprising sodium perchlorate, lithium chloride, guanidine HCl, guanidine thiocyanate, EDTA, EGTA, BAPTA, and/or adenine were prepared. Fresh samples of human urine were collected, spiked with 1 pg of gonococcal DNA, combined with one of the recited compositions, and incubated at room temperature. Aliquots were removed after 8 hours and tested by PCR for the presence of amplifiable gonococcal DNA. The PCR protocol was the same as described in Example 10. As illustrated in FIG. 15B, compositions with a chelator, a chelator enhancing component, and adenine stabilized gonococcal DNA in urine more effectively than compositions with fewer than all three components.

### EXAMPLE 17

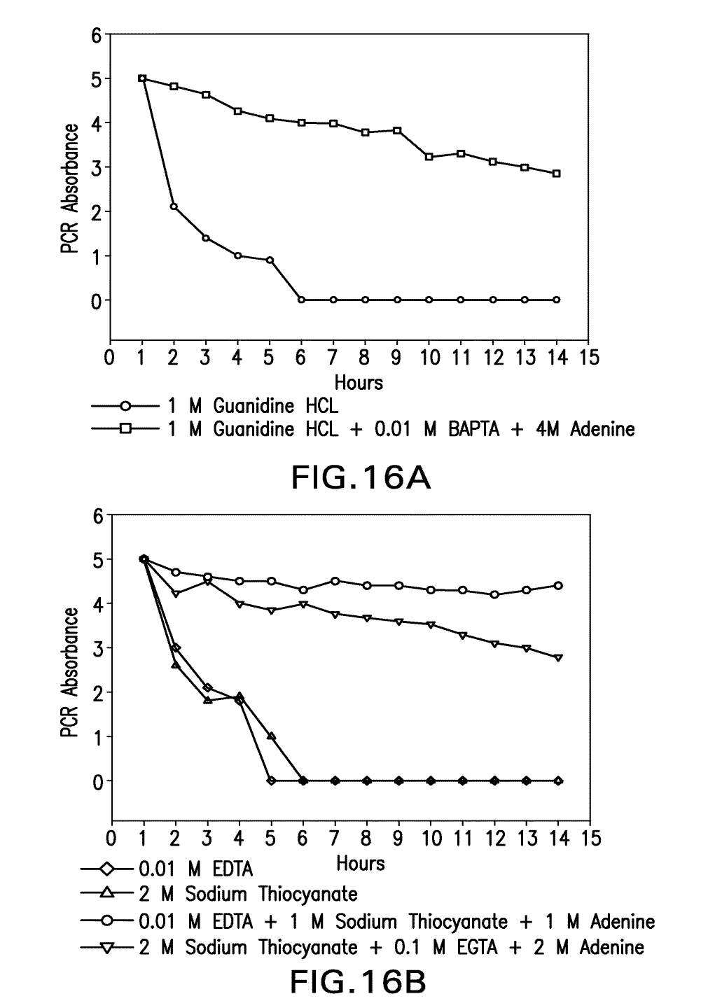

Compositions comprising sodium thiocyanate, guanidine HCl, EDTA, EGTA, BAPTA, and/or adenine were prepared. Fresh samples of human urine were collected, spiked with 1 pg of gonococcal DNA, combined with one of the recited compositions, and incubated at room temperature. Aliquots were removed after 8 hours and tested by PCR for the presence of amplifiable gonococcal DNA. The PCR protocol was the same as described in Example 10. As illustrated in FIG. 16, compositions with a chelator, a chelator enhancing component, and adenine stabilized gonococcal DNA in urine more effectively than compositions with just one of these components.

### EXAMPLE 18

A composition of the disclosure, according to some embodiments, may preserve and/or stabilize a whole cell (a “cell and/or macromolecule stabilizing composition”). In a specific example, 300 urine specimens were taken from patients with one or more of the following conditions: acute glomerulonephritis, acute pyelonephritis, nephrotic syndrome, acute tubular necrosis, cystitis, urinary tract neoplasia, and viral infection.

Within 10 minutes of collection, urine samples were either refrigerated (2-8° C.) or combined with a cell and/or macromolecule stabilizing composition (CSC) having 1 M sodium thiocyanate, 0.01 M EDTA, and 1 M adenine (9 mL urine+1 mL macromolecule stabilizing composition). Refrigerated samples were processed within 2 hours of collection.

As shown in Table 4, the preservation and/or stabilization of a variety of whole cells using a cell and/or macromolecule stabilizing composition was at least as good as refrigeration.

TABLE 4
Whole Cell Stabilization

| Cell Types | Refrigeration | CSC |
|---|---|---|
| Erythrocytes | Normal | Slightly crenated |
| Dysmorphic Erythrocytes | Detectable | Detectable |
| Leukocytes | Granular spheres + | Granular spheres |
| | Nuclear Seg. | (normal) + |
| | Crystal violet+ | Nuclear Seg. |
| | | Crystal violet+ |
| Pyuria (standardized slide) | >20 hpf | >20 hpf |
| Lymphocytes-Histiocytes | Detectable | Detectable (normal) |
| Renal Tubular Epithelial Cells | Detectable | Detectable (normal) |
| 5 fragments (when present) | Distinguishable | Distinguishable |
| Renal Tubular Epithelial Lipids | Detectable | Detectable |
| Maltese Cross Formation | | |
| Pigment in renal tubular epithelial cells | Prussian Blue+ | Prussian Blue+ |
| Squamous Epithelial Cells | Detectable (normal) | Detectable (normal) |
| Hyaline Casts (phase contrast detection) | Detectable | Detectable |
| Waxy Casts (brightfield microscopy) | Detectable, normal | Detectable, slightly |
| | morphology | broad |
| Granular Casts | Detectable | Detectable |
| Fatty Casts | Detectable | Detectable |
| Crystal Casts | Detectable | Detectable |
| Hemoglobin Blood Casts | Detectable | Very pale |
| Myoglobin Casts | Detectable | Not Detectable |
| Erythrocyte Casts | Detectable | Detectable |
| Leukocyte Casts: Brightfield microscopy | Detectable | Detectable |
| Phase contrast confirmation | Yes | Yes |
| Renal Tubular Epithelial Cell Casts (pap | Detectable | Variable |
| stain, phase contrast microscopy) | | |
| Bacteria | Detectable | Detectable |
| Crystal Acid Urine: | | |
| Calcium oxalates | Detectable | Detectable |
| Uric Acid | Detectable | Detectable |
| Crystalline Ureates | Detectable | Detectable |
| Crystal Alkaline Urine: | | |
| Amorphous Phosphates (CA-Mg) | Detectable | Detectable |
| Calcium Carbonate | Detectable | Detectable |
| Crystals Abnormal Urine: | | |
| Cystine | Detectable | Detectable |
| Sulfonamide crystals | Detectable | Detectable |

### EXAMPLE 19

During initial flow cytometry experiments, some red blood cells contacted with a composition consisting of 0.01 M EDTA and 1 M sodium thiocyanate appeared to be in good condition at day one, but were observed to form clumps at days 3-5 under the particular conditions tested. Samples containing clumped cells may be regarded to be more difficult to analyze by flow cytometry. Thus, improved formulations to stabilize cells for flow analysis were sought.

### EXAMPLE 20

A formulation was prepared that, according to some embodiments, may allow for the preservation of both red cell populations and white cell populations and the coexisting surface antigen markers on the white cells, with out the swelling and clumping that may be observed under some conditions.

An example embodiment of a composition may be prepared as follows:
1. Add lithium heparin to mixing container containing water. Mix until clear
2. Add Genelock stock chemistry to mixing container.
3. Add Adenine and mix until clear.
4. Add a surfactant (e.g., Butyryltri-n-hexyl Citrate). Mix for 10 min.
5. Add water to bring the total volume up to the desired final volume.
6. Mix for 15 min.
7. Filter (e.g., filter sterilize) to produce the final composition.
   According to some embodiments, a change in the chemistry may include substitution of lithium heparin for a citrate phosphate buffering system and an increase in the concentration of adenine. For example, a composition may include the following:

| | Amount/100 mL | |
|---|---|---|
| Genelock Stock Chemistry | 20 | mL |
| 1M sodium thiocyanate | | |
| 0.01M EDTA | | |
| Adenine | 0.03 | g |
| Lithium Heparin | 0.20 | g |
| Butyryltri-n-hexyl citrate (5% stock) | 0.5 | mL |
| QNS water to volume | | |

Blood was drawn on day one, combined with this composition at a preservative-to-blood ratio of 1:7, and aged at ambient temperatures (e.g., room temperature (RT)) for 3 days. The blood was subjected to a differential cell analysis on a Beckman coulter cell analyzer. The analysis showed excellent preservation of both white and red cell populations. The blood was pink and viscous with little change from the color of freshly collected blood. There was no visual evidence of a change in the blood and it is expected to be suitable for flow analysis.

### EXAMPLE 21

The following cell and/or macromolecule stabilizing compositions were prepared:

Composition 1: Molecular Whole Blood Tube

| Group A | g/L |
|---|---|
| Dextrose (monohydrate) | 31.9 |
| Sodium citrate (dihydrate) | 26.3 |
| Citric acid (anhydrous) | 3.27 |
| Monobasic sodium phosphate (monohydrate) | 2.22 |
| Adenine | 0.275 |

| Group B | | |
|---|---|---|
| Sodium thiocyanate | 405 | g/L |
| EDTA (0.1M) | 500 | mL/L |

| Composition 1 (A + B) | mL |
|---|---|
| Group A | 1000 |
| Group B | 100 |
| DIUF water | 200 |
| Total | 1300 |

| Composition FC | | |
|---|---|---|
| Sodium thiocyanate | 81 | g/L |
| EDTA (0.1M) | 100 | mL |
| Adenine | 0.30 | g/L |
| Sodium heparin | 2000 | mg/L |
| n-Butyryltri-n-hexyl citrate | 10 | mL/L |
| DIUF water | qs | |
| Total | 1000 | mL |

| Composition U-1 | | |
|---|---|---|
| Sodium thiocyanate | 81 | g/L |
| EDTA (0.1M) | 100 | mL |
| DIUF water | qs | |
| Total | 1000 | mL |

| Composition U-2 | | |
|---|---|---|
| Sodium thiocyanate | 81 | g/L |
| EDTA (0.1M) | 100 | mL |
| Adenine | 0.30 | g/L |
| DIUF water | qs | |
| Total | 1000 | mL |

| Composition S | | |
|---|---|---|
| Sodium thiocyanate | 8.1 | g/L |
| EDTA (0.1M) | 100 | mL |
| DMSO | 20 | mL/L |
| Glycerol | 25 | mL/L |
| Monobasic potassium phosphate | 3.93 | g/L |
| Tribasic potassium phosphate | 5.02 | g/L |

Fresh blood was combined with each of compositions 1 and FC at a preservative-to-blood ratio of 1:7, and aged at ambient temperatures (e.g., room temperature (RT)) for 3 days. After 24 hours, blood combined with composition U-1 clumped. By contrast, blood combined with composition FC had had no clumps after 72 hours. Viability was assessed using a trypan blue assay. Over 99% of white cells from blood combined with composition FC were intact (preserved) after 72 hours. Results are presented in Table 5.

| | Clumping | | | Viability | | |
|---|---|---|---|---|---|---|
| Composition | 24 hr | 48 hr | 72 hr | 24 hr | 48 hr | 72 hr |
| 1 | + | ++ | ++ | − | −− | −− |
| FC | −− | −− | −− | ++ | ++ | ++ |

Clumping: None (−−), Mild (+), Extensive (++)
Viability: None detected (−−), A few viable cells (−); 99% + Viable (++)

### EXAMPLE 22

Cell and/or macromolecule stabilizing compositions were prepared at ambient temperature and pressure by adding a chelator enhancing component (e.g., sodium thiocyanate) and deionized ultra-filtered (DTUF) water to a mixing container and then mixing for 10 minutes. Next, predissolved chelator (e.g., EDTA) was added to the mixing container and mixed for 10 minutes. A base (e.g., adenine) was then added to the container and mixed until a clear solution was obtained. If desired, a buffer (e.g., phosphate buffer) was added at this point. Finally, DIUF water was added to bring the volume in the mixing container up to the total desired volume and the solution was mixed for 10 minutes. The final solution was obtained by filter sterilizing the resulting mixture into a sterile container (e.g., a Nalgene bottle). The formula for several specific examples of cell and/or a macromolecule stabilizing compositions used in flow cytometry assays are elaborated in Table 5.

TABLE 5
Cell and/or a Macromolecule Stabilizing Compositions

| | | | | | T-8 | | | | | |
|---|---|---|---|---|---|---|---|---|---|---|
| | | | T-8 | | Phosphate | | T-8.25 | | T-8.5 | |
| | T-5 | | (1X) | | (1X) | | (1X) | | (1X) | |
| Sodium | 8.1 | g | 2.1 | g | 2.1 | g | 0.525 | g | 1.05 | |
| thiocyanate | | | | | | | | | | |
| 0.01M | 10 | mL | 10 | mL | 10 | mL | 10 | mL | 10 | mL |
| EDTA | | | | | | | | | | |
| n-butryryltri- | 1 | mL | None | | None | | None | | None | |
| hexyl-citrate | | | | | | | | | | |
| Adenine | 0.03 | g | 0.03 | g | 0.03 | g | 0.03 | g | 0.03 | g |
| Phosphate | None | | None | | | | None | | None | |
| buffer | | | | | | | | | | |
| Total | 100 | mL | 100 | mL | 100 | mL | 100 | mL | 100 | mL |
| Volume | | | | | | | | | | |

| | | | | | |
|---|---|---|---|---|---|
| pH | 5 | 8.0 | 6.5 | 8.25 | 8.5 |

### EXAMPLE 23

Cell and/or macromolecule stabilizing compositions were prepared at ambient temperature and pressure by adding an aliquot of USP purified water to an appropriately sized container, adding a chelator enhancing component (e.g., sodium thiocyanate), and then mixing. Next, predissolved chelator (e.g., EDTA) was added to the mixing container and mixed. Finally, USP purified water was added to bring the volume in the mixing container up to the total desired volume and the solution was mixed. The final solution (solution A) was obtained by filter sterilizing the resulting mixture into a sterile container (e.g., a Nalgene bottle).

Water was added to a second container. Dextrose was added and the composition was mixed until clear. Next, sodium citrate was added and the composition was mixed until clear. Citric acid was then added and the composition was again mixed until clear. Monobasic sodium phosphate was added and the composition was mixed until clear. Adenine was then added and the composition was mixed until clear. Finally, DUIF water was added to bring the volume in the mixing container up to the total desired volume and the solution was mixed. The final solution (solution B) was obtained by filter sterilizing the resulting mixture into a sterile container (e.g., a Nalgene bottle).

An aliquot of Solution B was added to a container followed by an aliquot of Solution A. The combined solutions were mixed (e.g., for 15 minutes) under ambient conditions. The formula for a specific example of a cell and/or a macromolecule stabilizing composition used in flow cytometry assays is elaborated in Table 6.

TABLE 6
Cell and/or a Macromolecule Stabilizing Compositions

| | Solution A | | Solution B | | T-10 | |
|---|---|---|---|---|---|---|
| Sodium thiocyanate | 10.02 | g | | | | |
| 0.01M EDTA | 50 | mL | | | | |
| Dextrose (monohydrate) | | | 3.19 | g | | |
| Sodium Citrate | | | 2.63 | g | | |
| (dihydrate) | | | | | | |
| Citric Acid (Anhydrous) | | | 0.327 | g | | |
| Monobasic Sodium | | | 0.220 | g | | |
| phosphate (monohydrate) | | | | | | |
| Adenine | | | 0.0275 | g | | |
| Solution A | | | | | 28 | mL |
| Solution B | | | | | 120 | mL |
| Total Volume | 100 | mL | 100 | mL | | |

| | |
|---|---|
| pH | 8.25 |

### EXAMPLE 24

Methods: Standard lymphocyte immunophenotyping by flow cytometry was performed on a Becton Dickenson FacsCalibur with beads for absolute count calibration. Using a lyse/no wash technique, whole blood was stained for CD3, CD4, CD8, and CD45 in one tube and CD16+56, CD19, and CD45 in another tube. By gating on forward scatter and CD45, lymphocytes were identified and 10,000 events counted. The percentage of lymphoctes that stain for each CD antigen and the absolute count of lymphoctes positive for each antigen were reported at 0, 24, 48, 72, 96, 120, 144, and 160 hours. Control tubes included EDTA (standard purple top) and heparin (standard green top) as well as EDTA and heparin in solution to account for any dilution effect of the test compositions. The stabilizing test reagents were prepared according to Examples 22 and 23. The pH of each composition is shown in Table 7. Flow parameters are shown in Table 8.

TABLE 7
pH of Compositions for Flow Cytometry

| | Strength/Dilution | | |
|---|---|---|---|
| pH | 0.25 X | 0.5 X | 1 X |
| T-8 | 8.0 | 8.0 | 8.0 |
| T-8 Phosphate | 6.5 | 6.5 | 6.5 |

TABLE 8
pH of Compositions for Flow Cytometry

| | | | | |
|---|---|---|---|---|
| Scatter Mode: | Forward | | | |
| | (FSC) | | | |
| | Side (SSC) | | | |
| Fluorescence: | FL1 | FITC | CD3 | T-cells |
| | FL2 | PE | CD8 | Suppressor cells |
| | | | CD16 + 56 | NK cells |
| | FL3 | PerCP | CD45 | White Cells |
| | FL4 | APC | CD4 | Helper cells |
| | | | CD19 | B-cells |

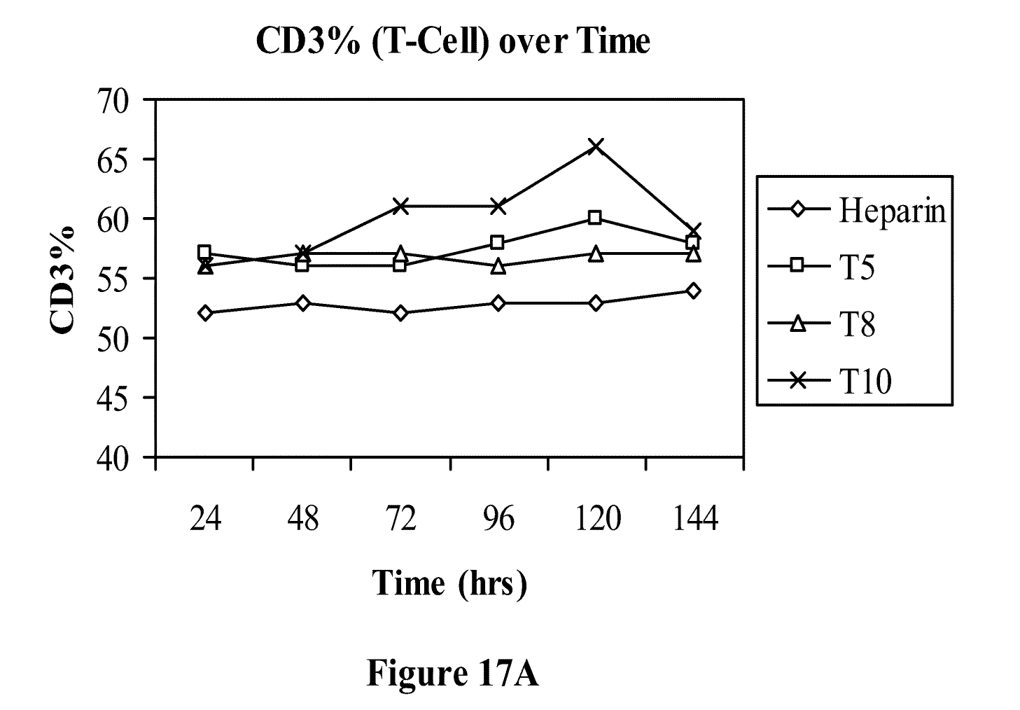

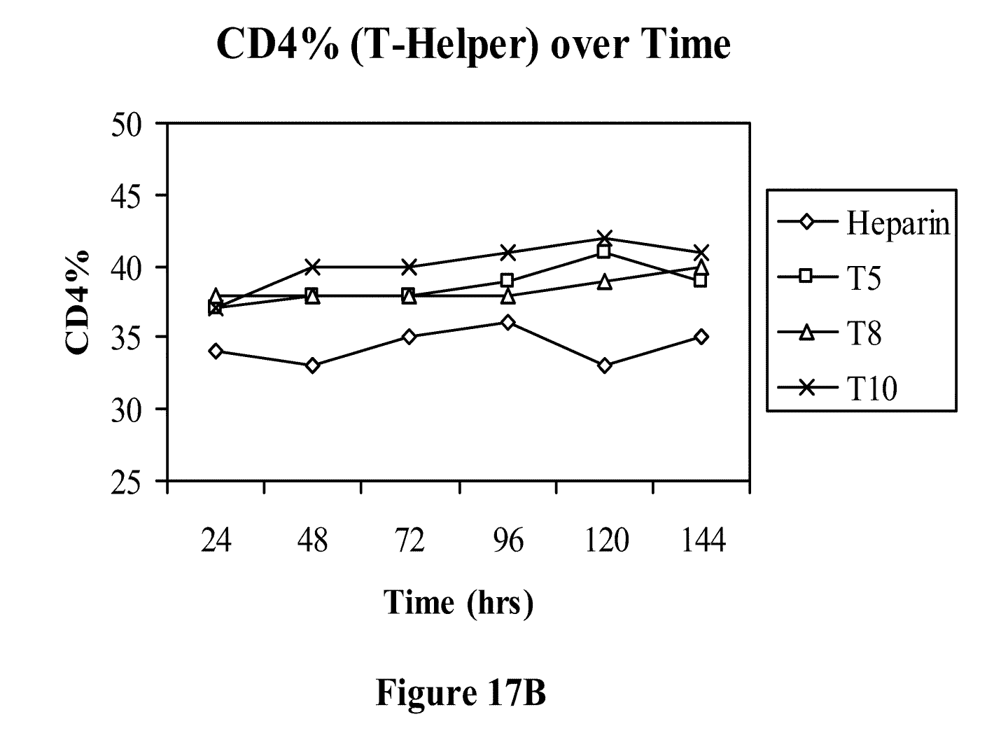

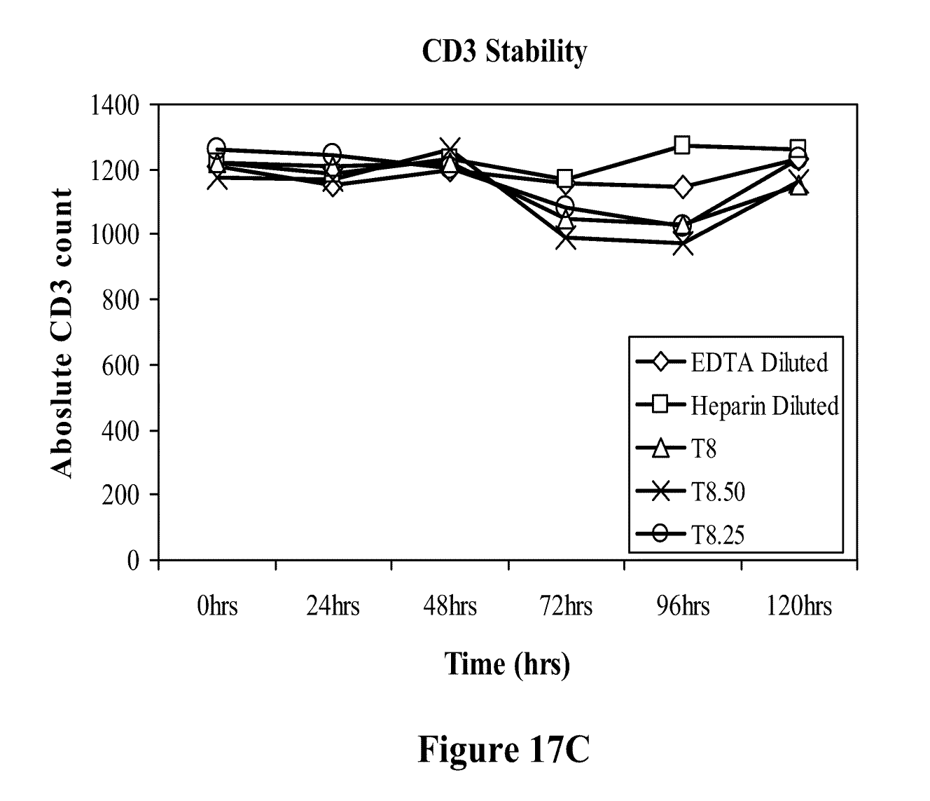

Results: By plotting the percentage and/or absolute count of the lymphocyte markers against time, the effectiveness of the different cell and/or macromolecule stabilizing compositions may be compared to current gold standard preservatives EDTA and heparin. For example, FIG. 17A plots CD3 percentage over time of the formulations compared to controls. Similarly, FIG. 17B plots CD4 percentage over time of the formulations compared to controls. The CD3 and CD4 percentages appear stable, even out to 160 hours, long past the recommended and accepted stability of both EDTA and heparin. In addition, the absolute counts of CD3 (number of CD3 cells per mL of blood) are stable out to 96 hours (FIG. 17C).

### EXAMPLE 25

RNA Methods: RNA was isolated at different time points from PAXgene™ tubes (which may include tetradecyltrimethylammonium oxalate and tartaric acid) or after hypotonic lysis of red blood cells using the Qiagen RNA Blood Mini kit. The isolated RNA was then quantified and its quality assessed using an Agilent 2100 BioAnalyzer using Pico cartridges (Agilent).

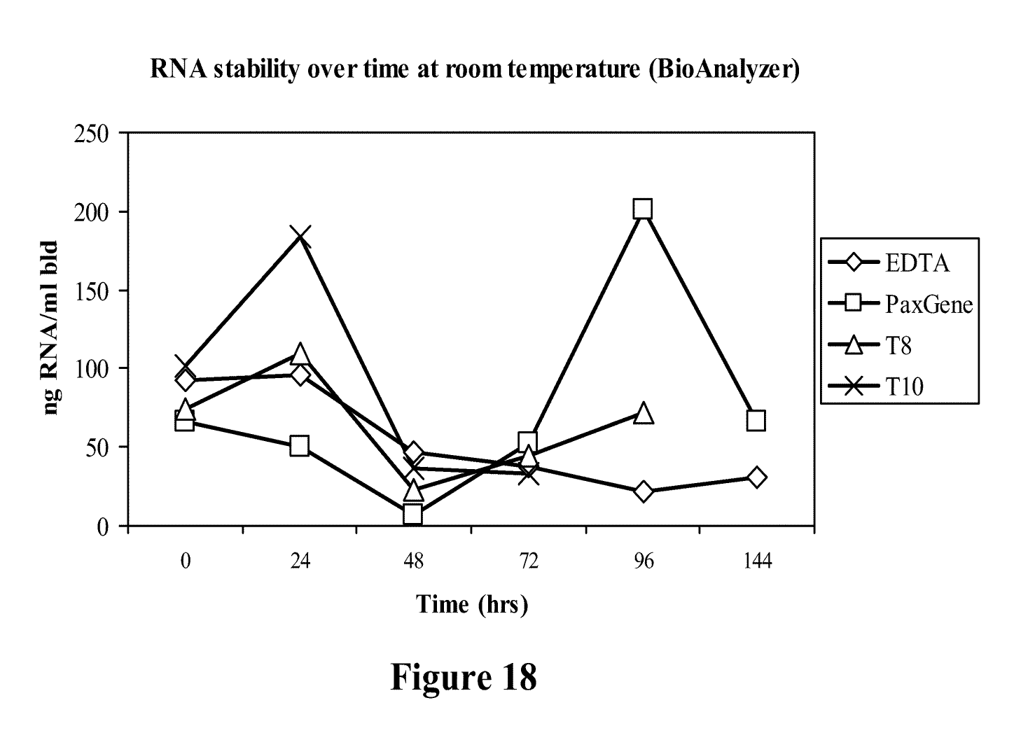

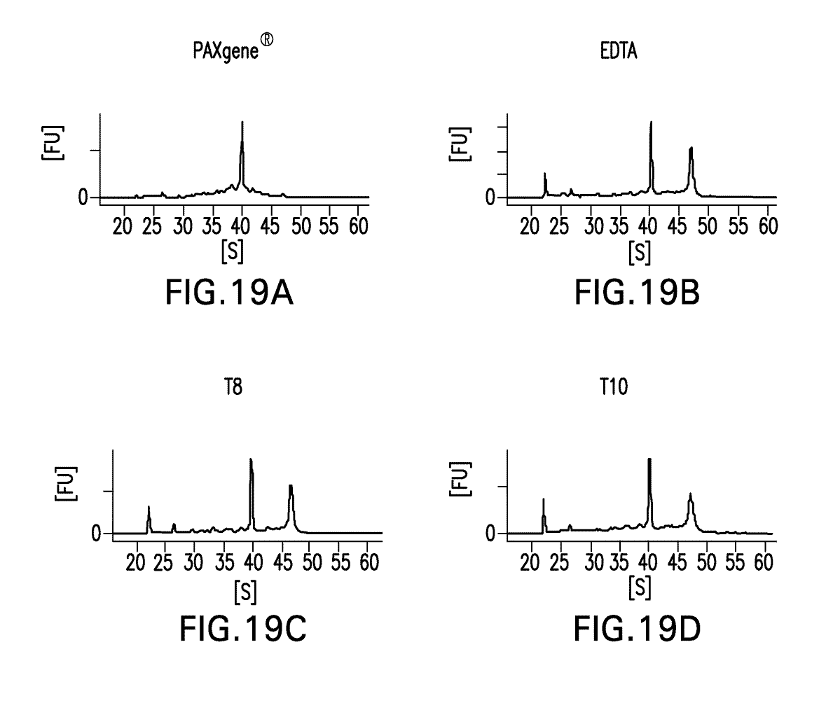

RNA Results: As shown in FIG. 18, at time points up to and including 48 hours, the RNA yield from samples preserved with T8 and T10 treatments was greater than PAXgene™ and approximately the same as the EDTA control. At 72 hours, the RNA yield from PAXgene™, T8, T10 and EDTA were all about the same. Sporadic clotting prevented analysis of some tubes after 72 hours. Not only was the amount of RNA obtained greater with cell and/or macromolecular stabilizing compositions according to the disclosure, but the quality of RNA from T8 and T10 was superior to the quality of PAXgene™ RNA and equivalent to the EDTA control. Quality data for RNA contacted with PAXgene™, EDTA, T8, and T10 at 72 hours is shown in FIGS. 19A, 19B, 19C, and 19D, respectively. The RNA integrity numbers (RIN) for these tests were 6.20 (19A), 7.90 (19B), 7.90 (19C), and 7.4 (19D). RNA quality may be assessed by the presence of two ribosomal RNA peaks on the right half of the trace. The larger ribosomal peak (farthest to the right) is absent in the PAXgene™ tube, indicating significant degradation.

What is claimed is:

1. A cell and/or macromolecule stabilizing composition, said composition comprising:
(a) a chelator selected from the group consisting of ethylenediaminetetraacetic acid (EDTA), [ethylenebis(oxyethylenenitrilo)]tetraacetic acid (EGTA), 1,2-bis(2-aminophenoxy)ethane-N,N,N′,N′-tetraacetic acid (BAPTA), and salts thereof;
(b) at least one chelator enhancing component selected from the group consisting of guanidine, lithium chloride, sodium salicylate, sodium perchlorate, and sodium thiocyanatc; and
(c) a base selected from the group consisting of a purine base and a pyrimidine base.

2. A cell and/or macromolecule stabilizing composition according to claim 1, wherein the concentration of the chelator is from about 0.1 mM to about 0.1 M.

3. A cell and/or macromolecule stabilizing composition according to claim 1, wherein the concentration of the at least one chelator enhancing component is from about 1 mM to about 5 M.

4. A cell and/or macromolecule stabilizing composition according to claim 1, wherein the concentration of the base is from about 0.1 mM to about 5 M.

5. A cell and/or macromolecule stabilizing composition according to claim 1, wherein the cell and/or macromolecule stabilizing composition is formulated as an aqueous solution.

6. A cell and/or macromolecule stabilizing composition according to claim 1, wherein the at least one chelator enhancing component is selected from the group consisting of sodium perchlorate, sodium thiocyanate, and lithium chloride.

7. A cell and/or macromolecule stabilizing composition according to claim 1, wherein the at least one chelator enhancing component is present in an amount of about 1 M.

8. A cell and/or macromolecule stabilizing composition according to claim 1, wherein the divalent metal chelator is present in an amount of about 1 mM.

9. A cell and/or macromolecule stabilizing composition according to claim 1, wherein the base is present in an amount of about 2 mM.

10. A cell and/or macromolecule stabilizing composition according to claim 1 further comprising a buffer.

11. A cell and/or macromolecule stabilizing composition according to claim 10, wherein the buffer comprises a compound selected from the group consisting of potassium acetate, sodium acetate, potassium phosphate, sodium phosphate, tris(hydroxyamino)methane, N-(2-hydroxyethyl)piperazine-N′-(2-ethanesulfonic acid), 3-(N-morpholino)propane sulfonic acid, 2-[(2-amino-2-oxoethyl)amino]ethanesulfonic acid, N-(2-acetamido)-2-iminodiacetic acid, 3-[(1,1-dimethyl-2-hydroxyethyl)amino]-2-propanesulfonic acid, N,N-bis(2-hydroxyethyl)-2-aminoethanesulfonic acid, N,N-bis(2-hydroxyethylglycine, bis-(2-hydroxyethyl)imino-tris(hydroxymethyl)methane, 3-(cyclohexylamino)-1-propanesulfonic acid, 3-(cyclohexylamino)-2-hydroxy-1-propanesulfonic acid, 2-(N-cyclohexylamino)ethanesulfonic acid, and combinations thereof.

12. A cell and/or macromolecule stabilizing composition according to claim 10, wherein the buffer comprises a compound selected from the group consisting of 3-[N,N-bis(2-hydroxyethyl)amino]-2-hydroxy-propanesulfonic acid, N-(2-hydroxyethylpiperazine)-N′-(3-propanesulfonic acid), N-(2-hydroxyethyl)piperazine-N′-(2-hydroxypropanesulfonic acid), 2-(N-morpholine)ethanesulfonic acid, triethanolamine buffer, imidazole, glycine, ethanolamine, 3-(N-morpholine)-2-hydroxypropanesulfonic acid, piperazine-N,N′-bis(2-ethanesulfonic acid), piperazine-N,N′-bis(2-hydroxypropanesulfonic acid), N-tris[(hydroxymethyl)methyl]-3-aminopropanesulfonic acid, 2-hydroxy-3-[tris(hydroxymethyl)methylamino]-1-propanesulfonic acid, N-[Tris(hydroxymethyl)methyl]-2-aminoethanesulfonic acid, N-[Tris(hydroxymethyl)methyl]glycine, 2-amino-2-methyl-1,3-propanediol, 2-amino-2-methyl-1-propanol, and combinations thereof.

13. A cell and/or macromolecule stabilizing composition according to claim 1 further comprising a cell.

14. A cell and/or macromolecule stabilizing composition according to claim 13, wherein the cell comprises a cell selected from the group consisting of a mammalian cell, a plant cell, a yeast cell, a bacterial cell, a virally-infected cell, a diseased cell, and combinations thereof.

15. A cell and/or macromolecule stabilizing composition according to claim 14, wherein the mammalian cell comprises a cell selected from the group consisting of an erythrocyte, a leukocyte, a lymphocyte, a histiocyte, an epithelial cell, and combinations thereof.

16. A cell and/or macromolecule stabilizing composition according to claim 1 further comprising a nucleic acid.

17. A cell and/or macromolecule stabilizing composition according to claim 16, wherein the nucleic acid comprises a poly nucleic acid selected from the group consisting of a ribonucleic acid, a deoxyribonucleic acid, and combinations thereof.

18. A cell and/or macromolecule stabilizing composition according to claim 1 further comprising a plasticizer.

19. A cell and/or macromolecule stabilizing composition according to claim 18, wherein the plasticizer comprises a citrated alcohol selected from the group consisting of triethyl citrate, acetyl triethyl citrate, tributyl citrate, acetyl tributyl citrate, trioctyl citrate, acetyl trioctyl citrate, trihexyl citrate, acetyl trihexyl citrate, butyryl trihexyl citrate, trimethyl citrate, and combinations thereof.

20. A cell and/or macromolecule stabilizing composition according to claim 18, wherein the plasticizer is butyryl trihexyl citrate.

21. A cell and/or macromolecule stabilizing composition according to claim 20, wherein the butyryl trihexyl citrate comprises n-butyryltri-n-hexyl citrate.

22. A cell and/or macromolecule stabilizing composition according to claim 18, wherein the concentration of the plasticizer is from about 0.1% (v/v) to about 10% (v/v).

23. A cell and/or macromolecule stabilizing composition according to claim 1 further comprising an anticoagulant.

24. A cell and/or macromolecule stabilizing composition according to claim 23, wherein the anticoagulant comprises a sulfated glycosaminoglycan selected from the group consisting of a heparin, a heparin salt, and combinations thereof.

25. A cell and/or macromolecule stabilizing composition according to claim 23, wherein the anticoagulant is a heparin salt selected from the group consisting of ammonium heparin, calcium heparin, lithium heparin, potassium heparin, sodium heparin, zinc lithium heparin, and combinations thereof.

26. A cell and/or macromolecule stabilizing composition according to claim 23, wherein the concentration of the anticoagulant is from about 200 mg/L to about 20 g/L.

27. A cell and/or macromolecule stabilizing composition according to claim 1 further comprising heparinase.

28. A method of preserving and/or stabilizing a cell, said method comprising:
contacting a cell with a cell and/or macromolecule stabilizing composition comprising
(a) a chelator selected from the group consisting of ethylenediaminetetraacetic acid (EDTA), [ethylenebis(oxyethylenenitrilo)]tetraacetic acid (EGTA), 1,2-bis(2-aminophenoxy)ethane-N,N,N′,N′-tetraacetic acid (BAPTA), and salts thereof; and
(b) at least one chelator enhancing component selected from the group consisting of guanidine, lithium chloride, sodium salicylate, sodium perchlorate, and sodium thiocyanate.

29. A method according to claim 28, wherein the cell and/or macromolecule stabilizing composition further comprises a base selected from the group consisting of a purine base and a pyrimidine base.

30. A method according to claim 28, wherein the cell comprises a cell selected from the group consisting of a mammalian cell, a virally-infected cell, a diseased cell, and combinations thereof.

31. A method according to claim 30, wherein the mammalian cell comprises a cell selected from the group consisting of an erythrocyte, a leukocyte, a lymphocyte, a histiocyte, an epithelial cell, and combinations thereof.

32. A method according to claim 30, wherein the mammalian cell comprises a human cell.

33. A method according to claim 28, wherein the concentration of the chelator is from about 0.1 mM to about 0.1 M.

34. A method according to claim 28, wherein the concentration of the at least one chelator enhancing component is from about 0.1 mM to about 0.5 M.

35. A method according to claim 29, wherein the concentration of the base is from about 0.1 mM to about 5 M.

36. A method according to claim 28, wherein the cell and/or macromolecule stabilizing composition is formulated as an aqueous solution.

37. A method according to claim 28, wherein the at least one chelator enhancing component is selected from the group consisting of sodium perchlorate, sodium thiocyanate, and lithium chloride.

38. A method according to claim 28 further comprising preserving an intracellular nucleic acid, wherein the nucleic acid selected from the group consisting of DNA, RNA, mRNA, and cDNA.

39. A method according to claim 38 wherein the nucleic acid is eukaryotic RNA.

40. A method according to claim 28, wherein the cell is present in a bodily fluid obtained from a human subject.

41. A method according to claim 40, wherein the volume ratio of cell and/or macromolecular composition to bodily fluid is from about 1:10 to about 10:1.

42. A method according to claim 40, wherein the contacting the cell with the cell and/or macromolecule stabilizing composition comprises adding the cell to the cell and/or macromolecule stabilizing composition.

43. A method according to claim 40, wherein the contacting the cell with the cell and/or macromolecule stabilizing composition comprises adding the cell and/or macromolecule stabilizing composition to the cell.

44. A method according to claim 40, wherein the cell and/or macromolecule stabilizing composition and the bodily fluid together form a stabilized bodily fluid composition that remains substantially free of clumps for up to about 5 days after the cell is contacted with the cell and/or macromolecule stabilizing composition.

45. A method according to claim 40, wherein the bodily fluid comprises a material selected from the group consisting of blood, blood serum, amniotic fluid, spinal fluid, conjunctival fluid, salivary fluid, vaginal fluid, stool, seminal fluid, and sweat.

46. A method according to claim 28, wherein the cell is a lymphocyte.

47. A method according to claim 28, wherein the cell retains at least about 80% of an extracellular marker for at least about 3 days after the contacting the cell with the cell and/or macromolecule stabilizing composition.

48. A method according to claim 47, wherein the extracellular marker is selected from the group consisting of CD2, CD3, CD4, CD5, CD7, CD8, CD10, CD13, CD14, CD16, CD19, CD20, CD21, CD22, CD33, CD34, CD45, CD56, CD57, and combinations thereof.

49. A method according to claim 28, wherein the cell retains at least about 95% of an extracellular marker at about 5 days after the contacting the cell with the cell and/or macromolecule stabilizing composition.

50. A method according to claim 28, wherein said cell and/or macromolecule stabilizing composition further comprises at least one compound selected from the group consisting of a long chain fatty acid, a long chain fatty ester, a long chain fatty alcohol, lithium, heparin, heparinase, butylhexylcitrate, and/or combinations thereof.

51. A method according to claim 29, further comprising contacting the cell with a flow cytometer.

52. A method according to claim 51, wherein the contacting the cell with a flow cytometer occurs up to about 2 days, up to about 3 days, up to about 4 days, up to about 5 days, up to about 6 days, or up to about 7 days after the contacting with a cell and/or macromolecule stabilizing composition.

53. A system for preserving a cell in a sample, said system comprising:
a sample container configured and arranged to receive and contain a sample comprising the cell; and
a cell and/or macromolecule stabilizing composition comprising
(a) a chelator selected from the group consisting of ethylenediaminetetraacetic acid (EDTA), [ethylenebis(oxyethylenenitrilo)]tetraacetic acid (EGTA), 1,2-bis(2-aminophenoxy)ethane-N,N,N′,N′-tetraacetic acid (BAPTA), and salts thereof;
(b) at least one chelator enhancing component selected from the group consisting of guanidine, lithium chloride, sodium salicylate, sodium perchlorate, and sodium thiocyanate; and
(c) a base selected from the group consisting of a purine base and a pyrimidine base.

54. A system according to claim 53 further comprising user instructions.

55. A system according to claim 53, wherein the sample container contains the cell and/or macromolecule stabilizing composition.

56. A system according to claim 53, wherein the cell and/or macromolecule stabilizing composition further comprises a solid, a liquid, or a hydrogel.

57. A system according to claim 53, wherein the sample container comprises at least one inner surface and at least one outer surface.

58. A system according to claim 53, wherein the cell and/or macromolecule stabilizing composition is a coating on the at least one inner surface.

59. A system comprising:
(a) a preserved cell;
(b) a cell and/or macromolecule stabilizing composition, said composition comprising:
(i) a chelator selected from the group consisting of ethylenediaminetetraacetic acid (EDTA), [ethylenebis(oxyethylenenitrilo)]tetraacetic acid (EGTA), 1,2-bis(2-aminophenoxy)ethane-N,N,N′,N′-tetraacetic acid (BAPTA), and salts thereof;
(ii) at least one chelator enhancing component selected from the group consisting of guanidine, lithium chloride, sodium salicylate, sodium perchlorate, and sodium thiocyanate; and
(iii) a base selected from the group consisting of a purine base and a pyrimidine base; and
(c) an analytical device.

60. A system according to claim 59, wherein the cell and/or macromolecule stabilizing composition further comprises: a plasticizer and an anticoagulant.

61. A system according to claim 59, wherein the analytical device is selected from the group consisting of a microscope, a plate-reader, a size-fractionating gel, a thermocycler, a flow cytometer, automated hematology analyzer, differential cell counter, cell sorter, beads, an affinity matrix, a spectrometer, and combinations thereof.
